SEQUENCE LISTING (54 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - DNA, 1326 bp, Homo sapiens
  source 1..1326: mol_type = other DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 3 - DNA, 603 bp, Homo sapiens
  source 1..603: mol_type = other DNA
atgctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 4 - protein, 200 aa, Homo sapiens
MLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - DNA, 600 bp, synthetic construct
  source 1..600: mol_type = other DNA
atgctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 6 - protein, 199 aa, synthetic construct
MLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGKKIETHKLTFRENAKAKTDHGAEIVYKSPVVSGDTSPRHLSNVSSTGSIDMVDSPQLATLADEVSASLAKQGL

SEQ ID NO: 7 - DNA, 603 bp, synthetic construct
  source 1..603: mol_type = other DNA
atgctgcagacagcccccgtgcccatgccagacctgaagaatgtcaagtccaagatcggctccactgagaacctgaagcaccagccgggaggcgggaaggtgcagataattaataagaagctggatcttagcaacgtccagtccgccgccgctgcgaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 8 - protein, 200 aa, synthetic construct
MLQTAPVPMPDLKNVKSKIGSTENLKHQPGGGKVQIINKKLDLSNVQSAAAAKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 9 - DNA, 603 bp, synthetic construct
  source 1..603: mol_type = other DNA
atgctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcagccgccgctgcgaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 10 - protein, 200 aa, synthetic construct
MLQTAPVPMPDLKNVKSKIGSTENLKHQPGGGKVQIINKKLDLSNVQSKCGSAAAAKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 11 - DNA, 603 bp, synthetic construct
  source 1..603: mol_type = other DNA
atgctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcgccgccgctgcgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 12 - protein, 200 aa, synthetic construct
MLQTAPVPMPDLKNVKSKIGSTENLKHQPGGGKVQIINKKLDLSNVQSKCGSKDNIAAAAGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 13 - DNA, 603 bp, synthetic construct
  source 1..603: mol_type = other DNA
atgctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccggccgccgctgcggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 14 - protein, 200 aa, synthetic construct
MLQTAPVPMPDLKNVKSKIGSTENLKHQPGGGKVQIINKKLDLSNVQSKCGSKDNIKHVPAAAAVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 15 - DNA, 603 bp, synthetic construct
  source 1..603: mol_type = other DNA
atgctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcagccgccaatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 16 - protein, 200 aa, synthetic construct
MLQTAPVPMPDLKNVKSKIGSTENLKHQPGGGKVQIINKKLDLSNVQSKCGSAANIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 17 - DNA, 603 bp, synthetic construct
  source 1..603: mol_type = other DNA
atgctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggatgccgccaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 18 - protein, 200 aa, synthetic construct
MLQTAPVPMPDLKNVKSKIGSTENLKHQPGGGKVQIINKKLDLSNVQSKCGSKDAAKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 19 - DNA, 603 bp, synthetic construct
  source 1..603: mol_type = other DNA
atgctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgccgccgctgcggcagctccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 20 - protein, 200 aa, synthetic construct
MLQTAPVPMPDLKNVKSKIGSTENLKHQPGGGKVQIINKKLDLSNVQSKCGSKDNIKHVPGGGSAAAAAAPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 21 - DNA, 603 bp, synthetic construct
  source 1..603: mol_type = other DNA
atgctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacgccgccgctgcggcagctaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 22 - protein, 200 aa, synthetic construct
MLQTAPVPMPDLKNVKSKIGSTENLKHQPGGGKVQIINKKLDLSNVQSKCGSKDNIKHVPGGGSVQIVYAAAAAAKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 23 - DNA, 603 bp, synthetic construct
  source 1..603: mol_type = other DNA
atgctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctggccgccgctgcggcagcttgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 24 - protein, 200 aa, synthetic construct
MLQTAPVPMPDLKNVKSKIGSTENLKHQPGGGKVQIINKKLDLSNVQSKCGSKDNIKHVPGGGSVQIVYKPVDLAAAAAACGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 25 - protein, 9 aa, synthetic construct
YPYDVPDYA

SEQ ID NO: 26 - protein, 22 aa, synthetic construct
GSGATNFSLLKQAGDVEENPGP

SEQ ID NO: 27 - protein, 10 aa, synthetic construct
EQKLISEEDL

SEQ ID NO: 28 - DNA, 33 bp, synthetic construct
  source 1..33: mol_type = other DNA
gccgccgctgcgaaggataatatcaaacacgtc

SEQ ID NO: 29 - DNA, 21 bp, synthetic construct
  source 1..21: mol_type = other DNA
ggactggacgttgctaagatc

SEQ ID NO: 30 - DNA, 31 bp, synthetic construct
  source 1..31: mol_type = other DNA
gccgccgctgcgaaacacgtcccgggaggcg

SEQ ID NO: 31 - DNA, 22 bp, synthetic construct
  source 1..22: mol_type = other DNA
tgagccacacttggactggacg

SEQ ID NO: 32 - DNA, 34 bp, synthetic construct
  source 1..34: mol_type = other DNA
gccgccgctgcgggaggcggcagtgtgcaaatag

SEQ ID NO: 33 - DNA, 22 bp, synthetic construct
  source 1..22: mol_type = other DNA
gatattatcctttgagccacac

SEQ ID NO: 34 - DNA, 32 bp, synthetic construct
  source 1..32: mol_type = other DNA
gccgccgctgcggtgcaaatagtctacaaacc

SEQ ID NO: 35 - DNA, 21 bp, synthetic construct
  source 1..21: mol_type = other DNA
cgggacgtgtttgatattatc

SEQ ID NO: 36 - DNA, 30 bp, synthetic construct
  source 1..30: mol_type = other DNA
gccgccaatatcaaacacgtcccgggaggc

SEQ ID NO: 37 - DNA, 22 bp, synthetic construct
  source 1..22: mol_type = other DNA
tgagccacacttggactggacg

SEQ ID NO: 38 - DNA, 27 bp, synthetic construct
  source 1..27: mol_type = other DNA
gccgccaaacacgtcccgggaggcggc

SEQ ID NO: 39 - DNA, 22 bp, synthetic construct
  source 1..22: mol_type = other DNA
atcctttgagccacacttggac

SEQ ID NO: 40 - DNA, 41 bp, synthetic construct
  source 1..41: mol_type = other DNA
gccgccgctgcggcagctccagttgacctgagcaaggtgac

SEQ ID NO: 41 - DNA, 25 bp, synthetic construct
  source 1..25: mol_type = other DNA
actgccgcctcccgggacgtgtttg

SEQ ID NO: 42 - DNA, 41 bp, synthetic construct
  source 1..41: mol_type = other DNA
gccgccgctgcggcagctaaggtgacctccaagtgtggctc

SEQ ID NO: 43 - DNA, 21 bp, synthetic construct
  source 1..21: mol_type = other DNA
gtagactatttgcacactgcc

SEQ ID NO: 44 - DNA, 40 bp, synthetic construct
  source 1..40: mol_type = other DNA
gccgccgctgcggcagcttgtggctcattaggcaacatcc

SEQ ID NO: 45 - DNA, 21 bp, synthetic construct
  source 1..21: mol_type = other DNA
caggtcaactggtttgtagac

SEQ ID NO: 46 - protein, 22 aa, synthetic construct
GSGATNFSLLKQAGDVEENPGP

SEQ ID NO: 47 - DNA, 53 bp, synthetic construct
  source 1..53: mol_type = other DNA
ggaagcggagctactaacttcagcctgctgaagcagtggcggccgctcgagtc

SEQ ID NO: 48 - DNA, 21 bp, synthetic construct
  source 1..21: mol_type = other DNA
tgctggatatctgcagaattc

SEQ ID NO: 49 - DNA, 53 bp, synthetic construct
  source 1..53: mol_type = other DNA
caggctggagacgtggaggagaaccctggacctcagtggcggccgctcgagtc

SEQ ID NO: 50 - DNA, 24 bp, synthetic construct
  source 1..24: mol_type = other DNA
cttcagcaggctgaagttagtagc

SEQ ID NO: 51 - DNA, 36 bp, synthetic construct
  source 1..36: mol_type = other DNA
taccgagctcggatccatgtacccatacgatgttcc

SEQ ID NO: 52 - DNA, 34 bp, synthetic construct
  source 1..34: mol_type = other DNA
gatatctgcagaattccaaaccctgcttggccag

SEQ ID NO: 53 - DNA, 36 bp, synthetic construct
  source 1..36: mol_type = other DNA
acctcagtggcggcctatggagcagaaactcatctc

SEQ ID NO: 54 - DNA, 33 bp, synthetic construct
  source 1..33: mol_type = other DNA
tagactcgagcggcctcacaaaccctgcttggc

(19) United States
(12) Patent Application Publication
(10) Pub. No.: US 2026/0240945 A1
(43) Pub. Date: Aug. 20, 2026

(54) INHIBITION OF TAU AGGREGATION

(71) Applicants: TOKYO METROPOLITAN INSTITUTE OF MEDICAL SCIENCE, Tokyo (JP); JUNTENDO EDUCATIONAL FOUNDATION, Tokyo (JP)

(72) Inventors: Shotaro SHIMONAKA, Tokyo (JP); Masato HASEGAWA, Tokyo (JP); Nobutaka HATTORI, Tokyo (JP); Yumiko MOTOI, Tokyo (JP)

(73) Assignees: TOKYO METROPOLITAN INSTITUTE OF MEDICAL SCIENCE, Tokyo (JP); JUNTENDO EDUCATIONAL FOUNDATION, Tokyo (JP)

(21) Appl. No.: 19/355,606

(22) Filed: Oct. 10, 2025

(30) Foreign Application Priority Data
Oct. 11, 2024 (JP) 2024-179384

(51) Int. Cl. A61K 38/17; A61P 25/28

(52) U.S. Cl. CPC A61K 38/1709; A61P 25/28

(57) ABSTRACT

Provided are a composition and the like for suppression of tau aggregation. An aspect of the present invention is a composition for suppression of tau aggregation, the composition including a variant of tau or a nucleic acid construct that expresses the variant.

### CROSS-REFERENCE TO RELATED APPLICATIONS

This application claims priority to Japanese Application No. 2024-179384, filed Oct. 11, 2024, the entire contents of which are hereby incorporated by reference.

### REFERENCE TO AN ELECTRONIC SEQUENCE LISTING

The contents of the electronic sequence listing (247322021000SEQLIST.xml; Size: 58,916 bytes; and Date of Creation: Oct. 9, 2025) is herein incorporated by reference in its entirety.

### FIELD OF THE DISCLOSURE

The present invention relates to inhibition of tau aggregation, and in particular relates to inhibition of seed-dependent tau aggregation.

### BACKGROUND OF THE DISCLOSURE

Neurodegenerative diseases such as Alzheimer's disease (AD), corticobasal degeneration (CBD), and progressive supranuclear palsy (PSP) are characterized by the accumulation of tau proteins (tau) in a lesion area of a patient's brain and are defined as a type of tauopathy (tau disease).

### CITATION LIST

Goedert M, Clavaguera F, Tolnay M. The propagation of prion-like protein inclusions in neurodegenerative diseases. Trends Neurosci. 2010 July; 33 (7): 317-25.

### SUMMARY OF THE DISCLOSURE

In recent years, the accumulation of tau, like prions, in a brain through repeated seed-dependent aggregate formation and intercellular propagation has been considered to be a cause of the onset and pathological progression of tauopathy.

Although research on intercellular propagation is still underway, it is expected that finding a method for suppressing the formation of protein aggregation that occurs in a seed-dependent manner will lead to the treatment and prevention of relevant diseases.

In view of the above-described problem, an object of an aspect of the present invention is to provide a composition, a method, and the like related to inhibition of tau aggregation.

In order to solve the above problem, the present invention includes, for example, the following aspects.
1) A composition for suppression of tau aggregation, the composition including a variant of tau or a nucleic acid construct that expresses the variant.
2) A variant of tau having at least one of the following mutations (2) to (4):
(2) a mutation(s) of an amino acid(s) corresponding to a 294th amino acid (lysine) and/or a 295th amino acid (aspartic acid) in the amino acid sequence represented by SEQ ID NO: 1;
(3) a mutation(s) of an amino acid(s) corresponding to one amino acid, two amino acids, three amino acids, four amino acids, five amino acids, or all of six amino acids in a region (SKVTSK) consisting of 316th to 321st amino acids in the amino acid sequence represented by SEQ ID NO: 1; and
(4) a mutation(s) of an amino acid(s) corresponding to one amino acid, two amino acids, three amino acids, four amino acids, five amino acids, or all of six amino acids in a region (KPVDLS) consisting of 311th to 316th amino acids in the amino acid sequence represented by SEQ ID NO: 1.
3) A method for suppressing seed-dependent tau aggregation, the method including a step of causing a variant of tau to co-exist with wild-type tau.

According to an aspect of the present invention, it is possible to provide a composition for inhibition of tau aggregation and utilization thereof. In particular, it is possible to provide a composition for inhibition of seed-dependent tau aggregation and utilization thereof.

### BRIEF DESCRIPTION OF THE DRAWINGS

FIG. 1 is a view illustrating a domain structure of tau protein. Human tau is a microtubule-binding protein consisting of 352 to 441 amino acids. A projection domain that regulates the distance between microtubules is present at the N-terminal, and a microtubule-binding domain that binds to microtubules is present at the C-terminal. The aggregation core sequences of AD, CBD, and PSP tau are located in the region of 274-380 aa according to the numbering of the longest 441-amino-acid isoform. The C-terminal fragment of Tau (243-441 aa) covers the entire aggregation core sequence.

FIG. 2 is a diagram illustrating seed-dependent aggregation and propagation of tau.

FIG. 3 is a view illustrating, regarding the Example of the present invention, the results indicating that tau^{Δ368} inhibits AD seed-dependent aggregation of tau^{WT}. A “HA-tau^{WT}/P2A/Myc-tau^{WT}” construct is cleaved to be co-expressed as “HA-tau^{WT}-P2A” and “Myc-tau^{WT}”. A “HA-tau^{Δ368}/P2A/Myc-tau^{WT}” construct is cleaved to be co-expressed as “HA-tau^{Δ368}-P2A” and “Myc-tau^{WT}”. As a result of a comparison of the aggregated Myc-tau^{wt} collected in Insol. fractions, the aggregated Myc-tau^{wt} band was significantly reduced in the sample in which HA-tau^{Δ368} was co-expressed.

FIG. 4 is a view illustrating, regarding the Example of the present invention, the results indicating that tau^{Δ368} inhibits AD seed-dependent aggregation of tau^{WT}. A “HA-tau^{WT}/P2A/Myc-tau^{WT}” construct is cleaved to be co-expressed as “HA-tau^{WT}-P2A” and “Myc-tau^{WT}”. A “HA-tau^{WT}/P2A/Myc-tau^{Δ368}” construct is cleaved to be co-expressed as “HA-tau^{WT}-P2A” and “Myc-tau^{Δ368}”. As a result of a comparison of the aggregated HA-tau^{WT} collected in Insol. fractions, the aggregated HA-tau^{WT} band was significantly reduced in the sample in which Myc-tau^{Δ368} was co-expressed.

FIG. 5 is a view illustrating, regarding the Example of the present invention, the results indicating that tau^{Δ368} does not influence CBD-dependent aggregation of tau^{WT}. A “HA-tau^{WT}/P2A/Myc-tau^{WT}” construct is cleaved to be co-expressed as “HA-tau^{WT}_P2A” and “Myc-tau^{WT}”. A “HA-tau^{Δ368}/P2A/Myc-tau^{WT}” construct is cleaved to be co-expressed as “HA-tau^{Δ368}-P2A” and “Myc-tau^{WT}”. As a result of a comparison of the aggregated Myc-tau^{WT} collected in Insol. fractions, a reduction of the aggregated Myc-tau^{WT} band was not observed even when HA-tau^{Δ368} was co-expressed.

FIG. 6 is a view illustrating, regarding the Example of the present invention, the results of a search for a site that causes a loss of the aggregation ability of tau against CBD seeds. The 294-297Ala mutation significantly decreased CBD seed-dependent aggregation.

FIG. 7 is a view illustrating, regarding the Example of the present invention, the results of a search for a site that causes a loss of the aggregation ability of tau against CBD seeds. The 294-295Ala mutation significantly decreased CBD seed-dependent aggregation.

FIG. 8 is a view illustrating, regarding the Example of the present invention, the results of a search for a site that causes a loss of the aggregation ability of tau against PSP seeds. All of the Ala substitutions significantly decreased PSP seed-dependent aggregation.

FIG. 9 is a view illustrating, regarding the Example of the present invention, the results indicating that Tau²⁹⁴⁻²⁹⁵ Ala exhibits specificity for CBD seeds and that tau^{316-321Ala} exhibits specificity for PSP seeds. The results on the left side in the drawing show that CBD seeds derived from three cases were used, and 294-295 Ala decreased aggregation in all of the cases. 316-321 Ala did not change aggregation. The results on the right side in the drawing show that PSP seeds derived from three cases were used, and 316-321 Ala decreased aggregation in all of the cases. 294-295 Ala did not change aggregation.

FIG. 10 is a view illustrating, regarding the Example of the present invention, the results indicating that tau²⁹⁴⁻²⁹⁵ Ala inhibits CBD seed-dependent aggregation of tau^{WT}. A “HA-tau^{WT}/P2A/Myc-tau^{WT}” construct is cleaved to be co-expressed as “HA-tau^{WT}_P2A” and “Myc-tau^{WT}”. A “HA-tau²⁹⁴⁻²⁹⁵Ala/P2A/Myc-tau^{WT}” construct is cleaved to be co-expressed as “HA-tau^{294-295Ala}-P2A” and “Myc-tau^{WT}”. As a result of a comparison of the aggregated Myc-tau^{WT} collected in Insol. fractions, the aggregated Myc-tau^{WT} band was significantly reduced in the sample in which HA-tau²⁹⁴⁻²⁹⁵Ala was co-expressed.

FIG. 11 is a view illustrating, regarding the Example of the present invention, the results indicating that tau^{316-321Ala} inhibits PSP seed-dependent aggregation of tau^{WT}. A “HA-tau^{WT}/P2A/Myc-tau^{WT}” construct is cleaved to be co-expressed as “HA-tau^{WT}-P2A” and “Myc-tau^{WT}”. A “HA-tau^{316-321Ala}/P2A/Myc-tau^{WT}” construct is cleaved to be co-expressed as “HA-tau^{316-321Ala}-P2A” and “Myc-tau^{WT}”. As a result of a comparison of the aggregated Myc-tau^{WT} collected in Insol. fractions, the aggregated Myc-tau^{WT} band was significantly reduced in the sample in which HA-tau^{316-321Ala} was co-expressed.

FIG. 12 is a view illustrating an amino acid sequence (SEQ ID NO: 1) of human wild-type tau and a base sequence (SEQ ID NO: 2) encoding the same. The bases/amino acids enclosed in boxes in the drawing are a region corresponding to Tau-CTF24 (243-441 amino acid fragment) illustrated in FIG. 13.

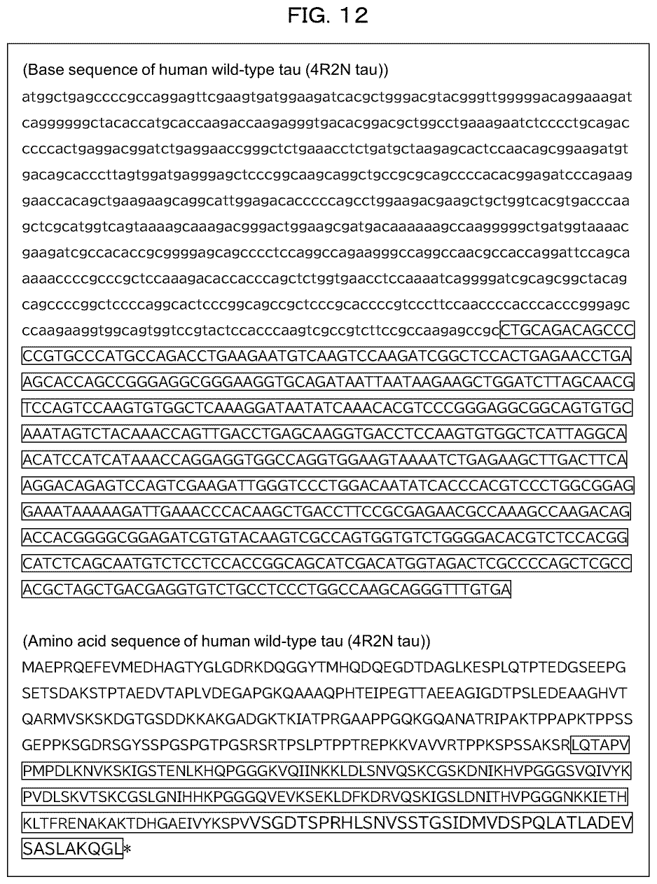

FIG. 13 is a view illustrating an amino acid sequence of the 243-441 amino acid fragment, Tau-CTF24, of human wild-type tau, and a base sequence encoding the same. A variant of tau in the Example corresponds to one obtained by introducing an amino acid mutation into this Tau-CTF24.

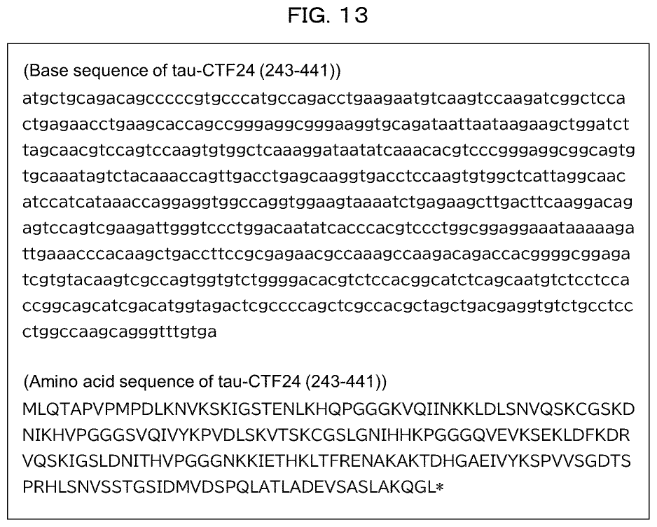

FIG. 14 is a view illustrating a base sequence and an amino acid sequence of a variant of tau in the Example. A deletion (loss) has occurred at the base/amino acid position in the underlined part in the drawing.

FIG. 15 is a view illustrating a base sequence and an amino acid sequence of a variant of tau in the Example. A substitution has occurred at the base/amino acid position enclosed in a box in the drawing.

FIG. 16 is a view illustrating a base sequence and an amino acid sequence of a variant of tau in the Example. A substitution has occurred at the base/amino acid position enclosed in a box in the drawing.

FIG. 17 is a view illustrating a base sequence and an amino acid sequence of a variant of tau in the Example. A substitution has occurred at the base/amino acid position enclosed in a box in the drawing.

FIG. 18 is a view illustrating a base sequence and an amino acid sequence of a variant of tau in the Example. A substitution has occurred at the base/amino acid position enclosed in a box in the drawing.

FIG. 19 is a view illustrating a base sequence and an amino acid sequence of a variant of tau in the Example. A substitution has occurred at the base/amino acid position enclosed in a box in the drawing.

FIG. 20 is a view illustrating a base sequence and an amino acid sequence of a variant of tau in the Example. A substitution has occurred at the base/amino acid position enclosed in a box in the drawing.

FIG. 21 is a view illustrating a base sequence and an amino acid sequence of a variant of tau in the Example. A substitution has occurred at the base/amino acid position enclosed in a box in the drawing.

FIG. 22 is a view illustrating a base sequence and an amino acid sequence of a variant of tau in the Example. A substitution has occurred at the base/amino acid position enclosed in a box in the drawing.

FIG. 23 is a view illustrating a base sequence and an amino acid sequence of a variant of tau in the Example. A substitution has occurred at the base/amino acid position enclosed in a box in the drawing.

### DETAILED DESCRIPTION OF THE DISCLOSURE

1. Variant of Tau and Nucleic Acid Construct that Expresses this Variant

A variant of tau in accordance with an embodiment of the present invention refers to a variant such that one or some of amino acids is/are mutated in an amino acid sequence that constitutes a primary structure of wild-type tau. This variant may also be referred to as tau variant or variant tau. An example of a mutation of an amino acid is a substitution of an amino acid and/or a loss (deletion) of an amino acid. The mutation is introduced by a genetic engineering technique.

In a comparison with the amino acid sequence (SEQ ID NO: 1) of wild-type tau, the position of an amino acid at which a mutation that influences tau aggregability (also referred to as “responsible mutation”) occurs is as shown in any of the following (1) to (4), and is preferably as shown in any of (1) to (3).

(1) A mutation of an amino acid corresponding to a 368th amino acid (asparagine) in an amino acid sequence represented by SEQ ID NO: 1. This responsible mutation is a mutation that influences tau aggregability specific to a seed of Alzheimer's disease (AD).

(2) A mutation(s) of an amino acid(s) corresponding to a 294th amino acid (lysine) and/or a 295th amino acid (aspartic acid) in the amino acid sequence represented by SEQ ID NO: 1. This responsible mutation is a mutation that influences tau aggregability specific to a seed of corticobasal degeneration (CBD).

(3) A mutation(s) of an amino acid(s) corresponding to one amino acid, two amino acids, three amino acids, four amino acids, five amino acids, or all of six amino acids in a region (SKVTSK) consisting of 316th to 321st amino acids in the amino acid sequence represented by SEQ ID NO: 1. This responsible mutation is a mutation that influences tau aggregability specific to a seed of progressive supranuclear palsy (PSP).

(4) A mutation(s) of an amino acid(s) corresponding to one amino acid, two amino acids, three amino acids, four amino acids, five amino acids, or all of six amino acids in a region (KPVDLS) consisting of 311th to 316th amino acids in the amino acid sequence represented by SEQ ID NO: 1. Preferably, a mutation(s) of an amino acid(s) corresponding to one amino acid, two amino acids, three amino acids, four amino acids, or all of five amino acids in a region (PVDLS) consisting of 312th to 316th amino acids in the amino acid sequence represented by SEQ ID NO: 1. This responsible mutation is a mutation that influences tau aggregability dependent on a seed of progressive supranuclear palsy (PSP).

For example, the variant of tau has, as the responsible mutation, only the mutation shown in (1) above, only the mutation(s) shown in (2) above, only the mutation(s) shown in (3) above, or only the mutation(s) shown in (4) above. For example, the variant of tau has, as the responsible mutation, two types (a combination of (1) and (2) above, a combination of (1) and (3) above, or a combination of (2) and (3) above) selected from among the mutations shown in (1) to (3) above. For example, the variant of tau has, as the responsible mutation, all of the mutations in (1), (2), and (3) above. For example, the variant of tau has, as the responsible mutation, two or three types selected from among the mutations shown in (1) to (4) above. For example, the variant of tau has, as the responsible mutation, all of the mutations in (1), (2), (3), and (4) above.

### Regarding the Responsible Mutation in (1) Above

An example of the mutation of the amino acid corresponding to the 368th amino acid (asparagine) in the amino acid sequence represented by SEQ ID NO: 1 is a deletion. Another example of the mutation is a substitution with a positively charged amino acid (arginine, lysine, or histidine). Still another example of the mutation is a substitution with a hydrophobic amino acid (alanine, valine, glycine, isoleucine, leucine, phenylalanine, proline, tryptophan, or tyrosine, and preferably, alanine, isoleucine, leucine, or valine).

### Regarding the Responsible Mutation in (2) Above.

An example of the mutation of the amino acid corresponding to the 294th amino acid (lysine) in the amino acid sequence represented by SEQ ID NO: 1 is a deletion. Another example of the mutation is a substitution with a negatively charged amino acid (aspartic acid or glutamic acid). Still another example of the mutation is a substitution with a hydrophobic amino acid (alanine, valine, glycine, isoleucine, leucine, phenylalanine, proline, tryptophan, or tyrosine, and preferably, alanine, isoleucine, leucine, or valine). Note that, in a case where the mutation of the amino acid corresponding to the later-described 295th amino acid (aspartic acid) in the amino acid sequence represented by SEQ ID NO: 1 is a substitution with a positively charged amino acid, the mutation of the amino acid corresponding to the 294th amino acid (lysine) can be preferably a deletion or a substitution with a hydrophobic amino acid.

An example of the mutation of the amino acid corresponding to the 295th amino acid (aspartic acid) in the amino acid sequence represented by SEQ ID NO: 1 is a deletion. Another example of the mutation is a substitution with a positively charged amino acid (arginine, lysine, or histidine). Still another example of the mutation is a substitution with a hydrophobic amino acid (alanine, valine, glycine, isoleucine, leucine, phenylalanine, proline, tryptophan, or tyrosine, and preferably, alanine, isoleucine, leucine, or valine). Note that, in a case where the mutation of the amino acid corresponding to the above-described 294th amino acid (lysine) in the amino acid sequence represented by SEQ ID NO: 1 is a substitution with a negatively charged amino acid, the mutation of the amino acid corresponding to the 295th amino acid (aspartic acid) can be preferably a deletion or a substitution with a hydrophobic amino acid.

### Regarding the Responsible Mutation in (3) Above

An example of the mutation(s) of the amino acid(s) corresponding to one amino acid, two amino acids, three amino acids, four amino acids, five amino acids, or all of six amino acids in the region (SKVTSK) consisting of the 316th to 321st amino acids in the amino acid sequence represented by SEQ ID NO: 1 is a deletion. Another example of the mutation(s) is a substitution, with a negatively charged amino acid (aspartic acid or glutamic acid), of an amino acid(s) corresponding to the 317th amino acid (lysine) and/or the 321th amino acid (lysine). Still another example of the mutation(s) is a substitution, with an amino acid other than serine, threonine, and tyrosine, of an amino acid corresponding to at least one amino acid selected from the 316th amino acid (serine), the 319th amino acid (threonine), and the 320th amino acid (serine). Yet another example of the mutation(s) is a substitution with a hydrophobic amino acid (alanine, valine, glycine, isoleucine, leucine, phenylalanine, proline, tryptophan, or tyrosine, and preferably alanine, isoleucine, leucine, or valine). However, an amino acid corresponding to the 318th amino acid (valine) may be unsubstituted (remain valine) or substituted with alanine.

### Regarding the Responsible Mutation in (4) Above

An example of the mutation(s) of the amino acid(s) corresponding to one amino acid, two amino acids, three amino acids, four amino acids, five amino acids, or all of six amino acids in the region (KPVDLS) consisting of the 311th to 316th amino acids in the amino acid sequence represented by SEQ ID NO: 1 is a deletion. Another example of the mutation(s) is a substitution, with a negatively charged amino acid (aspartic acid or glutamic acid), of the amino acid corresponding to the 311th amino acid (lysine). Still another example of the mutation(s) is a substitution with an amino acid other than serine, threonine, and tyrosine, of an amino acid corresponding to the 316th amino acid (serine). Yet another example of the mutation(s) is a substitution, with a positively charged amino acid (arginine, lysine, or histidine), of an amino acid corresponding to the 314th amino acid (aspartic acid). Further another example of the mutation(s) is a substitution with a hydrophobic amino acid (alanine, valine, glycine, isoleucine, leucine, phenylalanine, proline, tryptophan, or tyrosine, and preferably, alanine, isoleucine, leucine, or valine). However, an amino acid corresponding to the 312th amino acid (proline), an amino acid corresponding to the 313th amino acid (valine), or an amino acid corresponding to the 315th amino acid (leucine) may be unsubstituted (remain proline, valine, and leucine, respectively) or substituted with valine or alanine. Note that, in a case where the mutation of the amino acid corresponding to the 314th amino acid (aspartic acid) in the amino acid sequence represented by SEQ ID NO: 1 is a substitution with a positively charged amino acid, the mutation of the amino acid corresponding to the 311th amino acid (lysine) can be preferably a deletion or a substitution with a hydrophobic amino acid.

In a comparison with the amino acid sequence (SEQ ID NO: 1) of wild-type tau, the variant of tau may have an additional mutation (also referred to as “non-responsible mutation”), on the premise that the variant of tau has the above-described responsible mutation. The variant of tau is, for example, a form corresponding to a truncated form of wild-type tau. The truncated form of wild-type tau does not have, for example, a region on the N-terminal side of wild-type tau. Here, the region on the N-terminal side is, for example, all or contiguous positions in a region from the 1st position to 242nd position of wild-type tau, and is all or contiguous positions in a region of the 1st to 273rd positions (up to microtubule domain R1) of wild-type tau. In addition, the truncated form of wild-type tau does not have, for example, a region on the C-terminal side of wild-type tau. Here, the region on the C-terminal side is, for example, all or contiguous positions in a region from the 381st position to the 441st position of wild-type tau. The truncated form of wild-type tau does not have, for example, the above-described region on the N-terminal side of wild-type tau and/or the above-described region on the C-terminal region of wild-type tau. The truncated form of wild-type tau may be, for example, a truncated form having only a region corresponding to a microtubule-binding domain, R2, R³, or R4, a truncated form having only regions corresponding to the microtubule-binding domains R2 and R3, a truncated form having only regions corresponding to the microtubule-binding domains R3 and R4, or the like truncated form, on the premise that the truncated form of wild-type tau has the above-described responsible mutation.

Note that an “amino acid residue corresponding to a predetermined amino acid residue in the amino acid sequence represented by SEQ ID NO: 1” refers to 1) the predetermined amino acid residue itself in the amino acid sequence represented by SEQ ID NO: 1 and 2) an amino acid residue in another amino acid sequence corresponding to the predetermined amino acid residue in the amino acid sequence represented by SEQ ID NO: 1. In the case of 2), the “amino acid residue corresponding to a predetermined amino acid residue in the amino acid sequence represented by SEQ ID NO: 1” refers to an amino acid residue X in another amino acid sequence that is identified by homology analysis as corresponding to an amino acid residue X in the amino acid sequence represented by SEQ ID NO: 1. Note that the homology analysis is carried out by, for example, a method using pairwise sequence alignment, such as Needleman-Wunsch method and Smith-Waterman method, a method using multiple sequence alignment, such as ClustalW method, or the like method. In accordance with these methods, a person skilled in the art could use an amino acid sequence represented by SEQ ID NO: 1 as a reference sequence to understand the “corresponding amino acid residue” in another amino acid sequence to be analyzed. Examples of the another amino acid sequence to be analyzed include isoforms, homologs, and variants of the reference sequence. The analysis may be carried out under a default setting or may be carried out under a setting in which parameters have been appropriately changed from the default setting as necessary. This concept is also applied to amino acid sequences other than the amino acid sequence represented by SEQ ID NO: 1. Further, by reading amino acid residues as bases, this concept is also applied to any base sequence.

In an example of the variant of tau, an amino acid sequence excluding the above-described responsible mutation(s) exhibits, for example, a sequence identity of not less than 85%, not less than 90%, not less than 91%, not less than 92%, not less than 93%, not less than 94%, not less than 95%, not less than 96%, not less than 97%, not less than 98%, not less than 99%, or 100%, with respect to the corresponding region in the amino acid sequence represented by SEQ ID NO: 1. The variant of tau is preferably a variant based on the sequence of wild-type tau of a human, but may be a variant based on the sequence of wild-type tau of other animal (preferably a mammal), such as a mouse, a rat, or a pig.

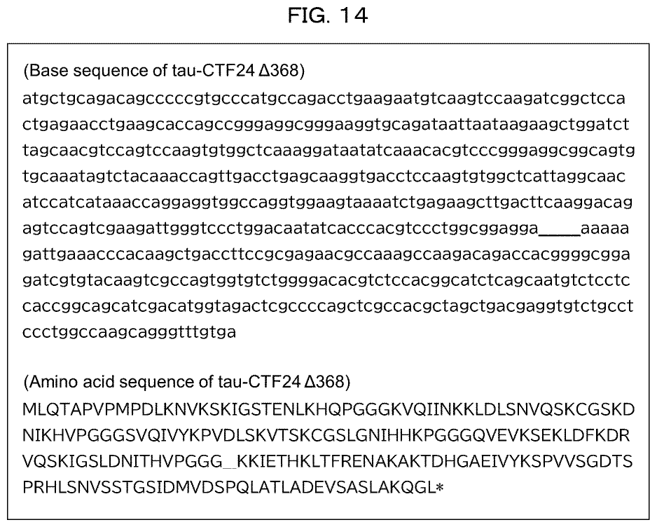

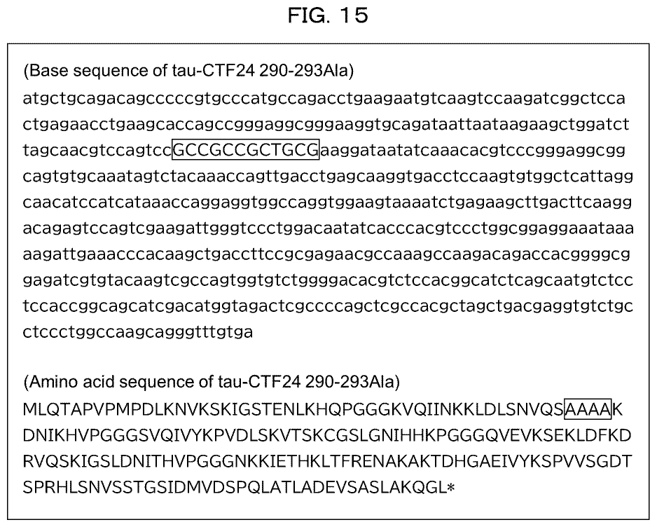

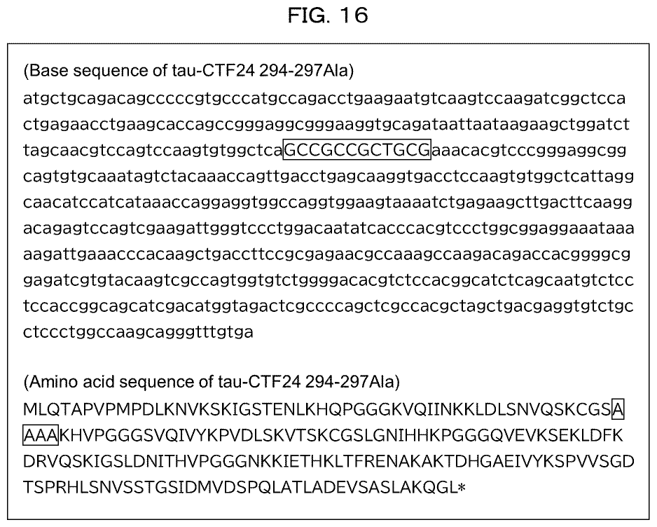

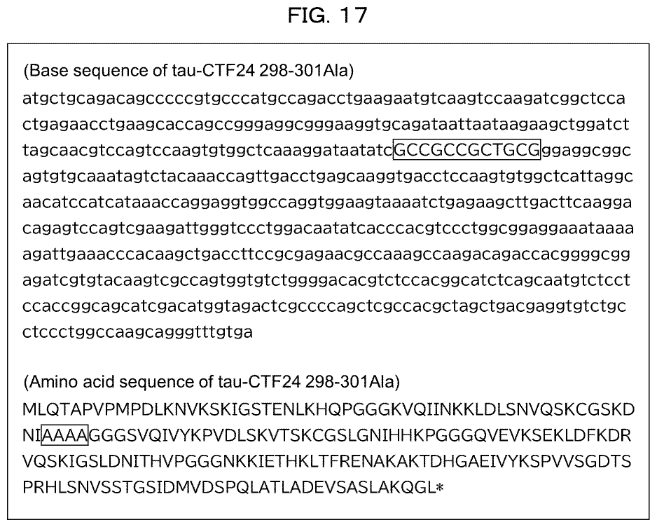

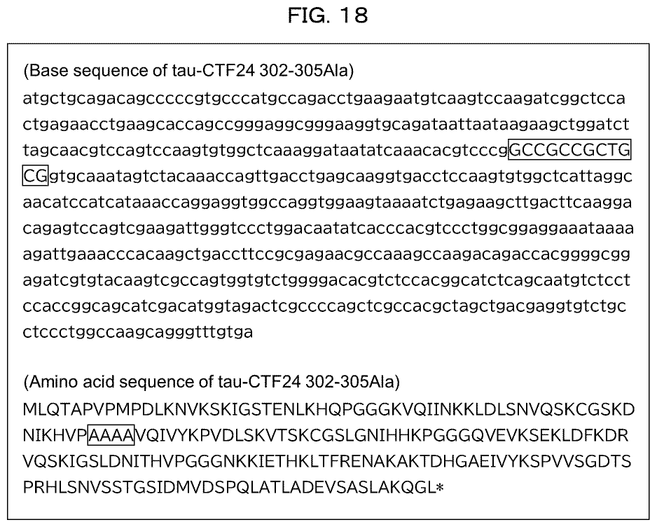

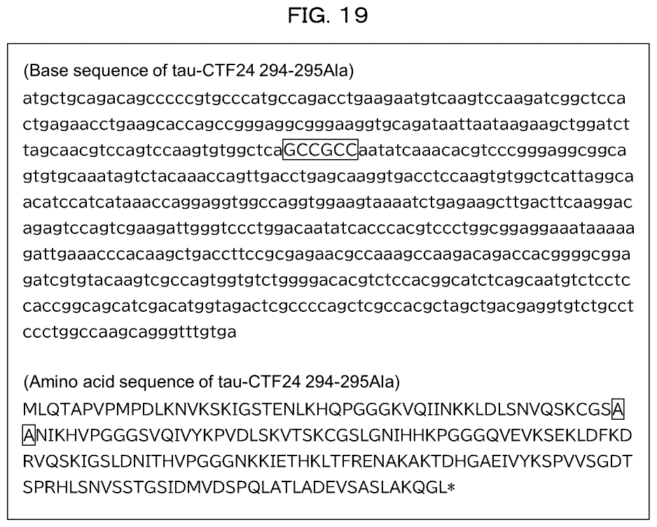

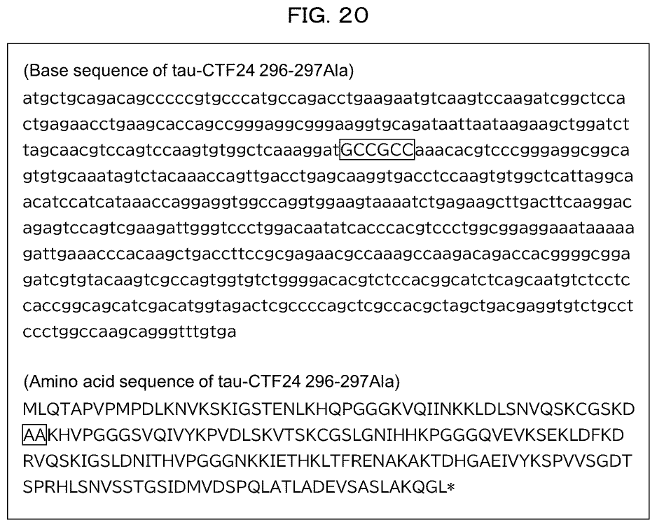

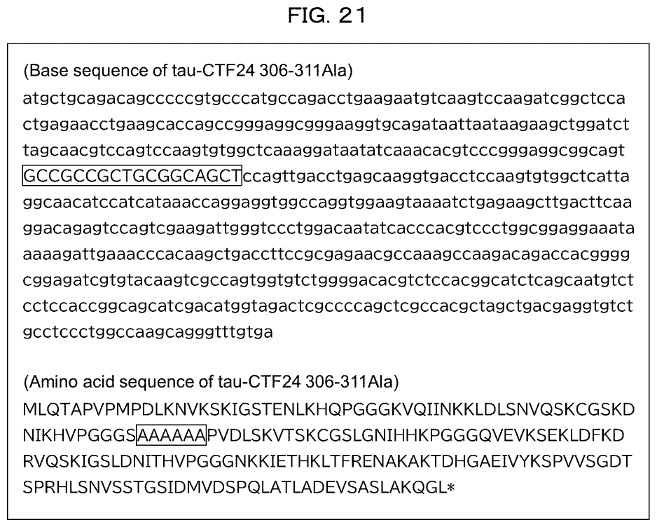

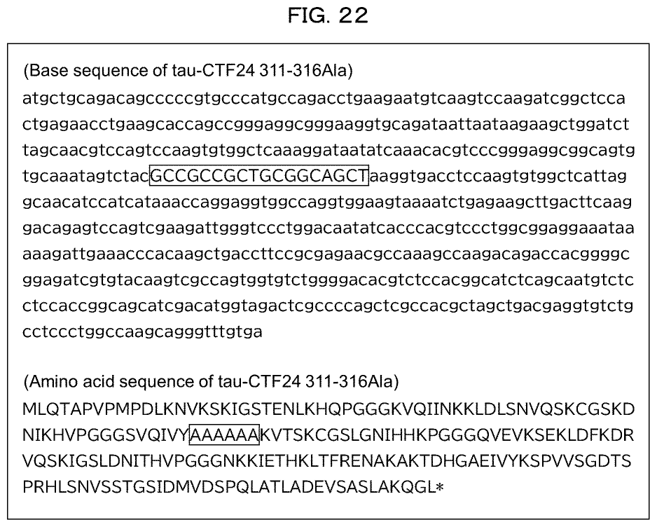

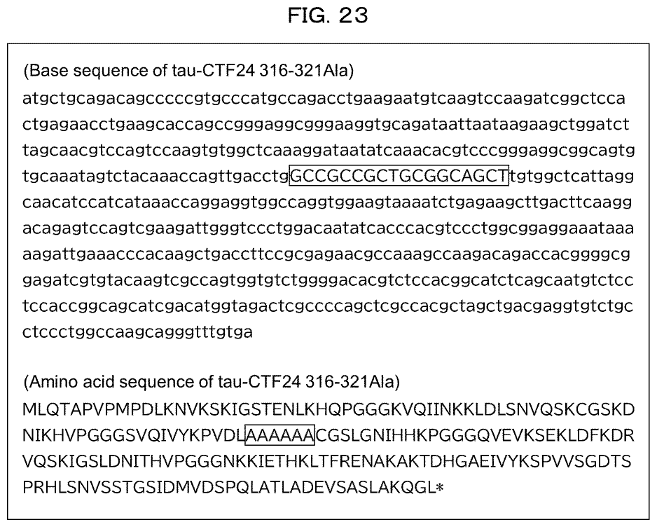

In another aspect, in an example of the variant of tau, an amino acid sequence excluding the above-described responsible mutation(s) exhibits, for example, a sequence identity of not less than 85%, not less than 90%, not less than 91%, not less than 92%, not less than 93%, not less than 94%, not less than 95%, not less than 96%, not less than 97%, not less than 98%, not less than 99%, or 100%, with respect to amino acid sequences of variants of tau (also illustrated in FIGS. 14 to 23) used in the Example.

In an example of the variant of tau, the amino acid length thereof is, for example, not more than 450 amino acids, not more than 400 amino acids, not more than 350 amino acids, not more than 300 amino acids, not more than 250 amino acids, not more than 200 amino acids, not more than 150 amino acids, not more than 120 or 110 amino acids, not more than 100 amino acids, or not more than 50 amino acids. In an example of the variant of tau, the amino acid length thereof is, for example, not less than 20 amino acids, not less than 30 amino acids, not less than 40 amino acids, not less than 50 amino acids, not less than 100 amino acids, or not less than 150 amino acids. The above-described examples of the upper limit values and lower limit values of the amino acid length of the variant of tau can be arbitrarily combined provided that the relationship of upper limit>lower limit is satisfied, and the amino acid length of the variant of tau can range from not less than the lower limit value to not more than the upper limit value.

For example, the variant of tau suppresses seed-dependent tau aggregation and accumulation, as described later, and can be used for treatment or prevention of tauopathies.

Nucleic Acid Construct that Expresses Variant of Tau

The nucleic acid construct in accordance with an embodiment of the present invention is a nucleic acid construct that expresses any of the above-described variants of tau. The type of nucleic acid constituting the nucleic acid construct is not particularly limited, and examples of the nucleic acid construct include a nucleic acid construct composed of DNA, a nucleic acid construct composed of RNA, and a nucleic acid construct composed of DNA and RNA. The nucleic acid construct may be constituted by including a non-natural nucleic acid as necessary.

An aspect of the nucleic acid construct in accordance with the present embodiment is a gene that expresses any of the above-described variants of tau. In a gene that expresses the variant of tau, a base sequence thereof excluding a region encoding the above-described responsible mutation exhibits, for example, a sequence identity of not less than 85%, not less than 90%, not less than 91%, not less than 92%, not less than 93%, not less than 94%, not less than 95%, not less than 96%, not less than 97%, not less than 98%, not less than 99%, or 100%, with respect to the corresponding region in the base sequence represented by SEQ ID NO: 2 (human tau gene). The variant of tau is preferably a variant based on the sequence of wild-type tau of a human, but may be a variant based on the sequence of wild-type tau of other animal (preferably a mammal), such as a mouse, a rat, or a pig.

In another aspect, in an example of the gene that expresses the variant of tau, a base sequence thereof excluding the region encoding the above-described responsible mutation exhibits, for example, a sequence identity of not less than 85%, not less than 90%, not less than 91%, not less than 92%, not less than 93%, not less than 94%, not less than 95%, not less than 96%, not less than 97%, not less than 98%, not less than 99%, or 100%, with respect to base sequences of genes that express variants of tau (also illustrated in FIGS. 14 to 23) used in the Example.

Another aspect of the nucleic acid construct in accordance with the present embodiment is an expression cassette which causes the above gene to be expressed. The expression cassette is preferably an expression vector, the type of which is selected as appropriate according to a host cell. The expression cassette is more preferably a virus vector such as a retrovirus vector, a lentivirus vector, an adenovirus vector, an adeno-related virus vector, an adeno-associated virus vector, a herpesvirus vector, and a vaccinia virus vector. Among these virus vectors, from the viewpoint of use in gene therapy of neurological disorders, an adeno-associated virus vector is preferable. In particular, from the viewpoint of use in gene therapy of neurological disorders, an adeno-associated virus vector such as a serotype 1 (AAV1) which exhibits tissue tropism toward the central nervous system, etc., a serotype 2 (AAV2) which exhibits broad tissue tropism, a serotype 5 (AAV5) which exhibits tissue tropism toward the central nervous system, etc., and a serotype 9 (AAV9) can be preferable.

The above expression cassette includes any expression regulatory region that is functional in an expression host. A promoter serving as the expression regulatory region is not limited to a particular type, but specific examples thereof can preferably include a promoter that provides high-level expression in mammals (in particular, human) or selectively functions in a specific tissue, such as a CAG promoter, a CMV promoter, or SYN (synapsin) I. In particular, the promoter can preferably be the CAG promoter. In the expression cassette, the promoter is provided on the upstream side of the gene.

The above expression cassette may further include, as necessary, a functional sequence that has an effect of, e.g., increasing the stability of mRNA produced through transcription, and that functions to, e.g., improve gene expression. Examples of the functional sequence include a woodchuck hepatitis virus posttranscriptional regulatory element (WPRE) sequence. In the expression cassette, the WPRE sequence is provided on the downstream side of the gene.

Another example of the functional sequence that may be included in the above expression cassette is a poly-A tail addition signal sequence. Examples of the poly-A tail addition signal sequence include the poly-A tail addition signal sequence of simian virus 40 and the poly-A tail addition signal sequence of human growth hormone. The poly-A tail addition signal sequence of human growth hormone can be more preferable. In the expression cassette, the WPRE sequence is provided on the downstream side of the gene.

The above expression cassette may further include inverted terminal repeat sequences (ITRs) and the like. The ITRs are provided in the expression cassette so as to sandwich the entire transcription unit (from the promoter to the poly-A tail addition signal sequence) therebetween.

The above-described expression cassette may constitute a nucleic acid construct such that a gene that expresses a variant of tau and another gene are expressed in a polycistronic manner under the control of an identical promoter. For example, a nucleic acid construct is constituted by interposing, between a gene that expresses a variant of tau and another gene, a sequence encoding an IRES element and/or a sequence encoding a 2A peptide. The type of the another gene is not particularly limited, but one example thereof is a gene that expresses wild-type tau. This makes it possible to easily produce an experimental system such that a variant of tau and wild-type tau are co-expressed in a host cell. Other example of the type of the another gene is a gene that expresses another variant of tau.

### 2. Composition for Suppression of Tau Aggregation

A composition in accordance with an embodiment of the present invention includes a variant of tau or a nucleic acid construct that expresses the variant. Note that the variant of tau and the nucleic acid construct are the ones described in the section [1. Variant of tau and nucleic acid construct that expresses this variant] above.

The composition in accordance with the present embodiment can be utilized as a composition for suppression of aggregation of tau (in particular, wild-type tau). This composition can be utilized particularly for the purpose of suppressing seed-dependent tau aggregation. Here, the “suppression of tau aggregation” refers to a phenomenon in which aggregation of tau (typically, wild-type tau) is suppressed in comparison with a case where the composition in accordance with the present embodiment is absent. The degree of suppression of tau aggregation is not particularly limited, but refers to a state in which, assuming that the amount of tau aggregation in the absence of the composition in accordance with the present embodiment is 100%, the amount of tau aggregation is, for example, 90% or less, 80% or less, 70% or less, 60% or less, 50% or less, 40% or less, 30% or less, 20% or less, 10% or less, or substantially no aggregation. Here, the suppression of tau aggregation may be a phenomenon under an in vitro (cell-free) environment or a phenomenon under an intracellular environment. The intracellular environment may be an environment within an isolated cell or may be an environment that is present in a body of a human or a non-human animal. The cell is preferably a nervous system cell and encompasses central nervous system cells, peripheral nervous system cells, glial cells, progenitor cells of these cells, and the like.

In addition, the seed-dependent tau aggregation refers to tau aggregation in which an abnormal protein molecule of tau is an aggregation nucleus (seed). The abnormal protein molecule of tau constitutes an aggregate. A tau aggregate may be, for example, a tau aggregate contained in a living body or an isolated tau aggregate. The tau aggregate can be one artificially constructed in vitro, one artificially constructed in a cell by a technique such as genetic recombination, or one naturally produced in a living body. For example, an artificially constructed wild-type tau can be degenerated and fibrillated, and a tau aggregate which serves as a seed can be obtained in a brain of a patient with a tauopathy (see also the Example).

The composition in accordance with an embodiment of the present invention is a composition for treatment or prevention of a tauopathy, i.e., a pharmaceutical composition. The pharmaceutical composition in accordance with an embodiment of the present invention preferably includes the nucleic acid construct described above. This pharmaceutical composition suppresses tau aggregation and accumulation in nervous system cells present in a body of a human or a non-human animal, as described above, and especially suppresses seed-dependent tau aggregation and accumulation. In addition, the pharmaceutical composition suppresses propagation of abnormally accumulated tau aggregates among nervous system cells, which occurs following seed-dependent tau aggregation and accumulation. As a result, the pharmaceutical composition exhibits the effects of preventing and/or treating a tauopathy, such as prevention of a tauopathy, halting of progression thereof, and delay of progression thereof. The scope of the present invention includes a method for treating and/or preventing a tauopathy in which an effective dose of this pharmaceutical composition is administered to an administration target.

A human or a non-human animal that is an administration target of the pharmaceutical composition is preferably any one selected from the group consisting of mammals including a human. The type of a mammal that is an administration target is not particularly limited, and examples thereof include: laboratory animals such as mice, rats, rabbits, guinea pigs, and primates other than humans; pet animals such as dogs and cats; farm animals such as cows and horses; and humans. Humans are particularly preferable. The human or the non-human animal that is an administration target is, for example, one that has an onset of a tauopathy (described later) or one that is at a stage prior to the onset of a tauopathy.

The tauopathy to be treated and/or prevented is not limited to a particular type, and broadly refers to neurodegenerative diseases characterized by abnormal structures and aggregation of tau in neuronal cells and glial cells. Tauopathies include, for example, Alzheimer's disease (AD), corticobasal degeneration (CBD), progressive supranuclear palsy (PSP), and the like. As described in the section [1. Variant of tau and nucleic acid construct that expresses this variant] above, among the four types of responsible mutations (1) to (4) identified herein, at least the three types of responsible mutations (1) to (3) are involved in suppression of seed-dependent aggregation according to the type of tauopathy. For example, a variant of tau having only the responsible mutation (1) above as the responsible mutation suppresses tau aggregation specific to a seed of Alzheimer's disease (AD), and can contribute to the treatment and/or prevention of Alzheimer's disease. A variant of tau having only the responsible mutation (2) above as the responsible mutation suppresses tau aggregation specific to a seed of corticobasal degeneration (CBD), and can contribute to the treatment and/or prevention of corticobasal degeneration. A variant of tau having only the responsible mutation (3) above as the responsible mutation suppresses tau aggregation specific to a seed of progressive supranuclear palsy (PSP), and can contribute to the treatment and/or prevention of progressive supranuclear palsy. As a matter of course, depending on the condition of the administration target of the pharmaceutical composition, a variant of tau having two or more types of mutations among the responsible mutations (1) to (4) or among the responsible mutations (1) to (3) may be used (or expressed), or two or more types of variants of tau having different responsible mutations may be used (or co-expressed).

A method of administering the pharmaceutical composition is not particularly limited. The pharmaceutical composition may be administered locally by a method such as injection (with use of a syringe, an injection pump, or the like), instillation into an eye, transdermal administration, or sublingual administration, or may be administered systemically by a method such as oral administration, intravascular administration such as intravenous administration or intraarterial administration, or enteral administration. In a preferable aspect of administration, the pharmaceutical composition is administered locally, in the vicinity of the nervous system targeted for therapy, etc.

The dosage (effective amount) of the pharmaceutical composition may be set as appropriate according to the age and sex of the human or animal as the administration target, the symptom, the administration route, the number of administrations, and the like. As necessary, an in vivo assay using the pharmaceutical composition can be carried out in advance to determine the dosage without requiring excessive experimentation.

The number of administrations of the pharmaceutical composition is not particularly limited provided that an effect is obtained, and can be set as appropriate, for example, according to the dosage, the administration route, the symptom, the age and sex of the human or animal.

A composition (including a pharmaceutical composition) in accordance with an embodiment of the present invention can include at least the above variant of tau or a nucleic acid construct that expresses the variant, and a carrier (for example, a pharmaceutically acceptable carrier). The carrier is not particularly limited, but preferably possesses the property of substantially not interfering with the functions of the variant of tau or the nucleic acid construct and the property of not causing any substantial adverse effects on the human or non-human animal as an administration target. The composition can preferably contain a liquid carrier such as water. As an example, the composition may be a liposomal preparation.

Examples of the component of the above composition further include, but are not particularly limited to, lubricants, preservatives, stabilizers, wetting agents, emulsifiers, osmotic pressure regulating salts, buffer agents, coloring agents, antioxidants, and viscosity modifiers.

### 3. Method for Suppressing Tau Aggregation, Etc.

A method in accordance with an embodiment of the present invention is a method for suppressing seed-dependent tau aggregation, the method including a step of causing a variant of tau to co-exist with wild-type tau. For example, the variant of tau is supplied with use of the composition described in the section [2. Composition for suppression of tau aggregation] above, or is expressed from a nucleic acid construct. The step of causing a variant of tau to co-exist with wild-type tau can be performed in an in vitro (cell-free) environment, in an intracellular environment, or in an in vivo (experimental animal such as a mouse or a rat) brain environment. For details of the method for suppressing tau aggregation, the description in the section [2. Composition for suppression of tau aggregation] above can be referred to.

In a method for designing a variant of tau in accordance with an embodiment of the present invention, a variant of tau is designed such that a responsible mutation is introduced into an amino acid residue located in a region that influences the aggregability of tau. The responsible mutation is the one described in the section [1. Variant of tau and nucleic acid construct that expresses this variant] above. The variant of tau designed in this manner is obtained by introducing a mutation, by a genetic engineering technique, based on a gene sequence encoding wild-type tau, and expressing the nucleic acid construct into which the mutation has been introduced. The obtained variant of tau is, as necessary, subjected to screening in order to confirm that the variant of tau has an ability to suppress seed-dependent tau aggregation. For example, the screening can be performed on the basis of whether seed-dependent aggregation of wild-type tau is suppressed when the obtained variant of tau is made to coexist with wild-type tau and a seed (see also the Example).

With the above embodiments considered together, the present invention can be summarized as follows.
1) A composition for suppression of tau aggregation, the composition including a variant of tau or a nucleic acid construct that expresses the variant.
2) The composition described in 1), wherein the variant of the tau has at least one of mutations shown in the following (1) to (4):
   (1) a mutation of an amino acid corresponding to a 368th amino acid (asparagine) in an amino acid sequence represented as an amino acid sequence (SEQ ID NO: 1) of wild-type tau;
   (2) a mutation(s) of an amino acid(s) corresponding to a 294th amino acid (lysine) and/or a 295th amino acid (aspartic acid) in the amino acid sequence represented by SEQ ID NO: 1;
   (3) a mutation(s) of an amino acid(s) corresponding to one amino acid, two amino acids, three amino acids, four amino acids, five amino acids, or all of six amino acids in a region (SKVTSK) consisting of 316th to 321st amino acids in the amino acid sequence represented by SEQ ID NO: 1; and
   (4) a mutation(s) of an amino acid(s) corresponding to one amino acid, two amino acids, three amino acids, four amino acids, five amino acids, or all of six amino acids in a region (KPVDLS) consisting of 311th to 316th amino acids in the amino acid sequence represented by SEQ ID NO: 1.
3) The composition described in 1) or 2), wherein the composition suppresses seed-dependent tau aggregation.
4) The composition described in 3), wherein the composition suppresses tau aggregation dependent on a seed of Alzheimer's disease, suppresses tau aggregation dependent on a seed of corticobasal degeneration, or suppresses tau aggregation dependent on a seed of progressive supranuclear palsy.
5) The composition described in any of 1) to 4), wherein the composition is for treating or preventing a tauopathy.
6) The composition described in any of 1) to 5), wherein the variant of the tau is one type or two or more types of variants (two types of variants, three types of variants, four types of variants, or five types of variants) of the tau selected from the group consisting of a variant having the mutation shown in (1) above, a variant having the mutation shown in (2) above, a variant having the mutation shown in (3) above, a variant shown in (4) above, and a variant shown in the following (5):
(5) a mutation(s) of an amino acid(s) corresponding to one amino acid, two amino acids, three amino acids, four amino acids, five amino acids, or all of six amino acids in a region (VQIVYK) consisting of 306th to 311th amino acids in the amino acid sequence represented by SEQ ID NO: 1.

7) A variant of tau having at least one of the following mutations (2) to (4):
(2) a mutation(s) of an amino acid(s) corresponding to a 294th amino acid (lysine) and/or a 295th amino acid (aspartic acid) in the amino acid sequence represented by SEQ ID NO: 1;
(3) a mutation(s) of an amino acid(s) corresponding to one amino acid, two amino acids, three amino acids, four amino acids, five amino acids, or all of six amino acids in a region (SKVTSK) consisting of 316th to 321st amino acids in the amino acid sequence represented by SEQ ID NO: 1; and
(4) a mutation(s) of an amino acid(s) corresponding to one amino acid, two amino acids, three amino acids, four amino acids, five amino acids, or all of six amino acids in a region (KPVDLS) consisting of 311th to 316th amino acids in the amino acid sequence represented by SEQ ID NO: 1.

8) A nucleic acid construct encoding the variant of tau described in (7).

9) A method for suppressing seed-dependent tau aggregation, the method including a step of causing a variant of tau to co-exist with wild-type tau.

10) In 9) above, the variant of tau is supplied with use of the composition described in any of 1) to 6), or is expressed from the nucleic acid construct that constitutes the composition described in any of 1) to 6).

11) In 9) or 10) above, the variant of tau is the variant of tau described in 7), or is expressed from the nucleic acid construct described in 8).

12) In any of 9) to 11) above, the step of causing the variant of tau to co-exist with wild-type tau can be performed in an in vitro (cell-free) environment, in an intracellular environment, or in vivo.

The present invention is not limited to the embodiments, but can be altered by a skilled person in the art within the scope of the claims. The present invention also encompasses, in its technical scope, any embodiment derived by combining technical means disclosed in differing embodiments.

### EXAMPLES

The following will describe an Example of the present invention.

### Example 1: Tau Aggregation Inhibition Method Using Artificially Mutation-Introduced Tau that Distinguishes Among Tau Strains

Neurodegenerative diseases such as Alzheimer's disease (AD), corticobasal degeneration (CBD), and progressive supranuclear palsy (PSP) are characterized by the accumulation of tau proteins (tau) in a lesion area of a patient's brain and are defined as a type of tauopathy (tau disease). The accumulation of tau, like prions, in a brain through repeated seed-dependent aggregate formation and intercellular propagation has been considered to be a cause of the onset and pathological progression of tauopathy. Thus, it is expected that achieving suppression of tau aggregation will lead to an effective treatment method for tauopathy.

Here, the inventors of the present invention found in a cellular model that AD seed-dependent aggregation of tau^{WT} (wild-type) was potently inhibited by co-expressing tau^{Δ368}, which is an artificially mutated tau that ceases to undergo AD seed-dependent aggregation. This indicates that the tau^{Δ368} interfered and inhibited the process of AD seed-dependent aggregation of other tau molecules, and it was found that the tau^{Δ368} had the potential to be utilized as a “therapeutic protein” which functions to inhibit tau aggregation. In addition, in order to design an artificially mutated tau for addressing CBD seed-dependent aggregation and PSP seed-dependent aggregation, the inventors of the present invention searched for artificial mutations that ceased to undergo CBD seed-dependent aggregation and PSP seed-dependent aggregation, and consequently identified tau^{294-295Ala} for CBD and tau^{316-321Ala} for PSP. Subsequently, in a co-expression system with tau^{WT} in a cellular model, it was confirmed that these variants suppressed CBD seed-dependent aggregation and PSP seed-dependent aggregation of tau^{WT}, respectively.

The present Example showed that various types of artificially mutated tau found here have the potential of serving as a tau-targeting disease-modifying drug that suppresses tau aggregation in typical tauopathies, namely AD, CBD, and PSP.

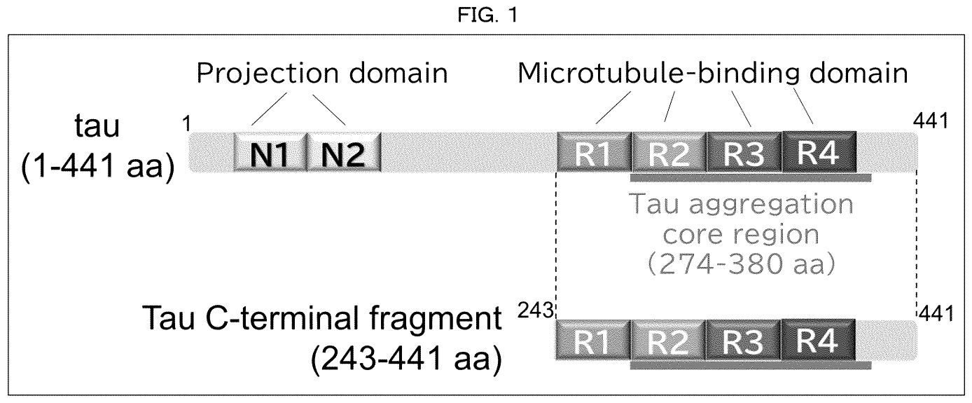

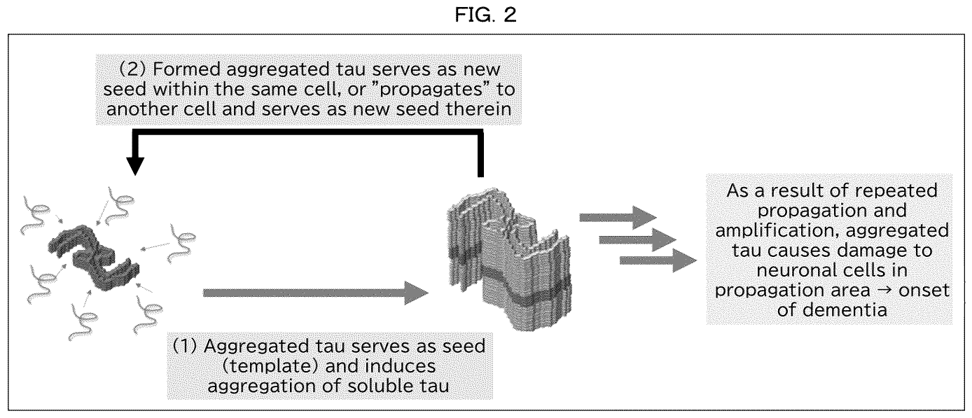

In neurodegenerative diseases, notably Alzheimer's disease (AD), such as corticobasal degeneration (CBD) and progressive supranuclear palsy (PSP), accumulation of abnormally aggregated tau proteins (tau) (FIG. 1) is observed in a lesion area of a brain and is collectively referred to as “tauopathy”. In recent years, the microstructures of tau aggregates in AD, CBD, and PSP have been determined, and it has been confirmed that the tau aggregates have distinctly different structural polymorphisms (tau strains). These tau aggregates, like prions, serve as aggregation nuclei (seeds) to induce structural changes in unaggregated tau, extend while incorporating the unaggregated tau into their own structures, and propagate between cells to cause progressive neuronal cell death (FIG. 2). In different types of tauopathies, tau aggregates with different structures are accumulated in different regions of a brain. Thus, clinical manifestations vary from one disease to another. Therefore, in order to establish a definitive treatment method for these tauopathies, an aggregation inhibition method capable of accommodating tau aggregation patterns that vary from one disease to another is necessary.

The inventors have previously found that tau^{Δ368}, in which the 368th asparagine residue is deleted, does not aggregate even in the presence of AD patient-derived tau seeds (AD seeds) (Shimonaka et al., J Biol Chem. 2020; 295 (41): 13996-14014). The tau^{Δ368} can be said to be a variant that lacks only the aggregation ability against AD seeds because the tau^{Δ368} aggregates without any problem in the presence of CBD patient-derived tau seeds and PSP patient-derived tau seeds (CBD seeds and PSP seeds). Thereafter, an experiment was conducted with the aim of causing no-deletion tau (tau^{wt}; WT: wild type; wild-type) to aggregate with use of an AD seed as a nucleus in the presence of tau^{Δ368}. As a result of the experiment, not only aggregation of tau^{Δ368} but also aggregation of tau^{WT} were significantly suppressed. This result demonstrated that tau^{Δ368} exhibits a property of serving as an aggregation inhibitor such that tau^{Δ368} not only does not undergo aggregation by AD seeds but also positively acts and inhibits AD seed-dependent aggregation of other tau molecules.

In the present Example, it is shown that respective tau aggregations in AD, CBD, and PSP can be inhibited by designing artificially mutated tau that exhibits an aggregation suppression effect specific to each tau strain, and the purpose of the present Example is to establish a foothold for clinical application as a definitive treatment method for tauopathy that targets tau as a treatment target.

Human tau is a microtubule-binding protein consisting of 352 to 441 amino acid residues (FIG. 1). Since the core region of aggregated tau seen in AD, CBD, and PSP patients' brains corresponds to 274-380 aa, the previous study conducted by the inventors used a tau C-terminal fragment of 243-441 aa covering this region, and the inventors continued to use this tau fragment in the present Example. When tau is expressed in SH-SY5Y using a vector for cellular expression, and an AD seed extracted from an AD patient's brain is introduced into the cell, the AD seed becomes an aggregation nucleus to aggregate the expressed tau, resulting in the formation of an insoluble tau aggregate in a surfactant. The tau aggregate can be collected in a surfactant-insoluble fraction of a cell lysate in isolation with non-aggregated soluble tau and can be quantified on western blotting (cellular model of seed-dependent tau aggregation).

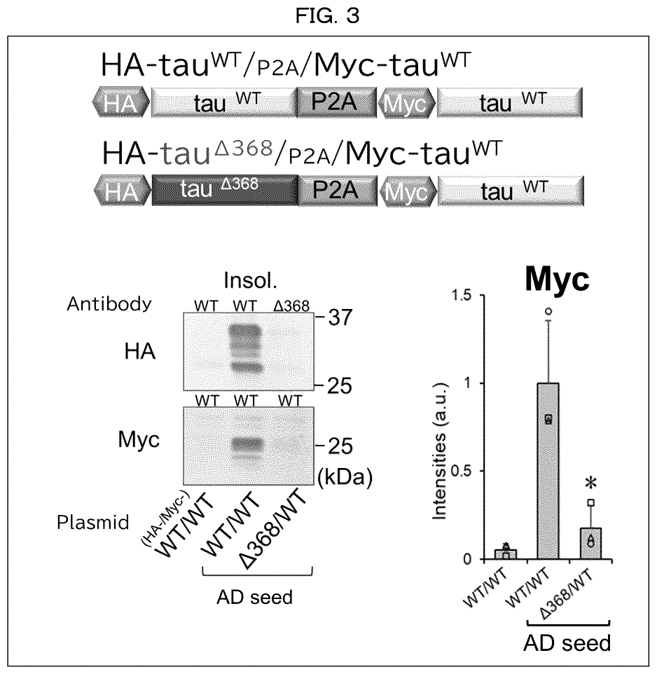

The inventors constructed a co-expression vector “pcDNA3-HA-tau^{Δ368}/P2A/Myc-tau^{WT}” by incorporating, downstream of a vector for cellular expression “pcDNA3-HA-tau^{Δ368} (HA: hemagglutinin tag sequence YPYDVPDYA)”, a “P2A sequence (GSGATNFSLLKQAGDVEENPGP)” that self-cleaves during intracellular translation, and “Myc-tau^{WT} (Myc: Myc tag sequence EQKLISEEDL)”. By transfecting this co-expression vector into SH-SY5Y cells, a nascent protein in the middle of translation is cleaved into “HA-tau^{Δ368}-P2A” and “Myc-tau^{WT}”, resulting in a pseudo-co-expression system (FIG. 3). The inventors also constructed “pcDNA3-HA-tau^{WT}/P2A/Myc-tau^{WT}” to co-express tau^{wt}, caused both of the vectors to each express in SH-SY5Y cells, added AD seeds, and cultured the cells for 3 days. Thereafter, the cells were collected, homogenized in A68 buffer, and ultracentrifuged, and then pellets were collected. Next, the pellets were homogenized in A68 buffer containing 1% Triton-X100 (surfactant) and ultracentrifuged, and then the pellets were collected. Finally, the pellets were homogenized in A68 buffer containing sarkosyl (surfactant) and ultracentrifuged, and then the pellets were collected. A 2×SDS sample buffer was added to the pellets, followed by homogenization. The resulting homogenate was regarded as a surfactant-insoluble fraction (insol. fraction). Western blotting of insol. fractions was performed using an anti-Myc-tag antibody to quantify the band intensity of aggregated Myc-tau^{WT}. As a result, the band observed in the sample in which HA-tau^{WT} was co-expressed was remarkably reduced in the sample in which HA-tau^{Δ368} was co-expressed.

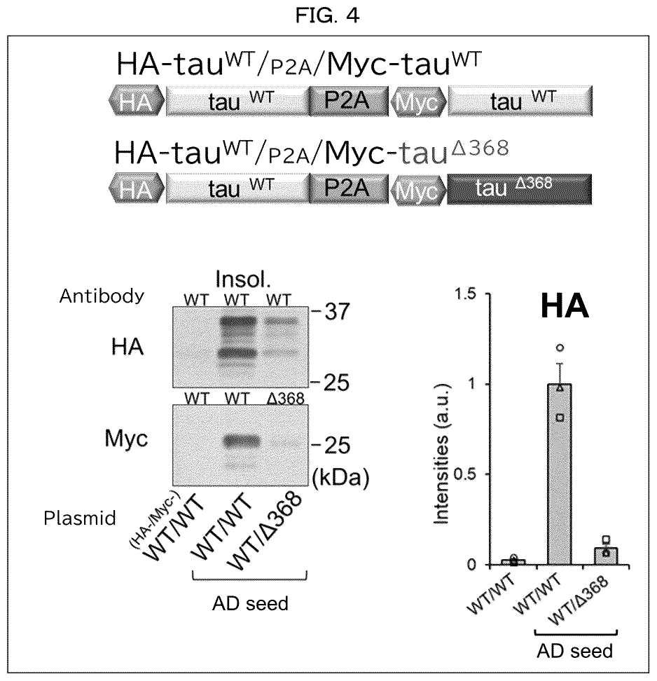

Next, a construct with a reversed arrangement order (“pcDNA3-HA-tau^{WT}/P2A/Myc-tau^{Δ368}”) was prepared by incorporating WT on the upstream HA side of P2A and incorporating 4368 on the downstream Myc side, and a similar experiment was conducted (FIG. 4). Western blotting of insol. fractions was performed using an anti-HA-tag antibody to quantify the band intensity of aggregated HA-tau^{WT}. As a result, the band observed in the sample in which Myc-tau^{WT} was co-expressed was remarkably reduced in the sample in which Myc-tau^{Δ368} was co-expressed.

The results of these two experiments both indicate that the AD seed-induced aggregation of tau^{WT} was strongly inhibited in the presence of tau^{Δ368}, and suggest that tau^{Δ368} interfered in and suppressed the process of aggregation of tau^{WT}.

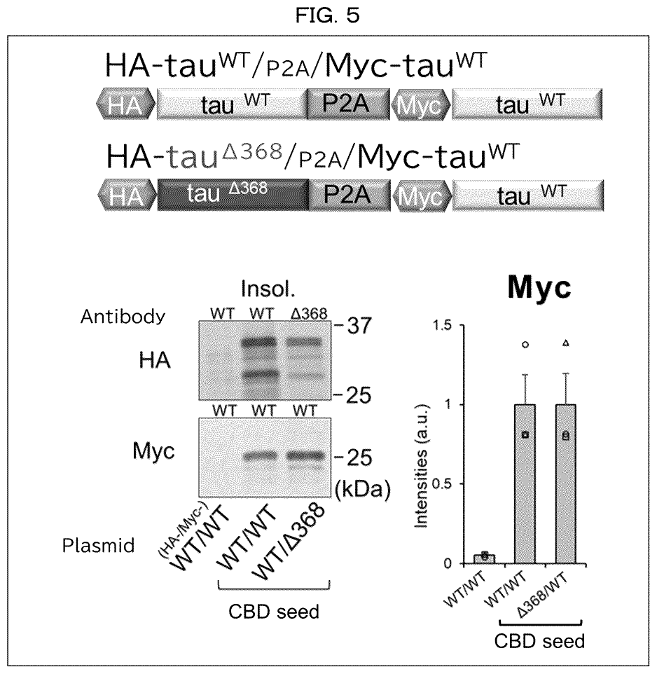

To confirm whether tau^{Δ368} inhibits aggregation induced by tau seeds other than AD seeds, CBD seeds were introduced into cells expressing “pcDNA3-HA-tau^{WT}/P2A/Myc-tau^{WT}” and “pcDNA3-HA-tau^{Δ368}/P2A/Myc-tau^{WT}”, and the cells were cultured for 3 days (FIG. 5). Thereafter, insol. fractions were prepared in the same manner, and Western blotting was performed to quantify the aggregated Myc-tau^{WT} band.

As a result, it was revealed that Myc-tau^{WT} aggregated to similar extents by being induced by the CBD seeds, regardless of whether HA-tau^{WT} or HA-tau^{Δ368} was co-expressed. This result indicates that, unlike in the case of the AD seeds, the CBD seed-induced aggregation of tau^{WT} was not inhibited even in the presence of tau^{Δ368} Therefore, it was suggested that the aggregation suppression effect of tau^{Δ368} is exerted specifically only on tau aggregation of the AD strain.

The aggregation suppression effect of tau^{Δ368} is not exerted on tau strains other than AD strain. Thus, for example, for the treatment of tauopathies such as CBD and PSP, it is necessary to design new artificially mutated tau tailored for these strains. The inventors attempted to identify, like A368 for AD seeds, mutation sites that cause loss of aggregation ability against CBD and PSP seeds. As a method, first, a series of variants into which Ala mutations of several residues in length were introduced were prepared so as to comprehensively cover sites of interest in the tau sequence. Next, these variants were expressed in SH-SY5Y cells, and, after aggregation was induced by CBD and PSP seeds, aggregation for each variant was quantified by Western blotting of the insol. fractions, thereby identifying variants in which aggregation was markedly reduced. The mutation sites possessed by these variants were regarded as candidates for strain-specific sequences. Finally, from among the candidates, those capable of distinguishing between a CBD seed and a PSP seed were selected.

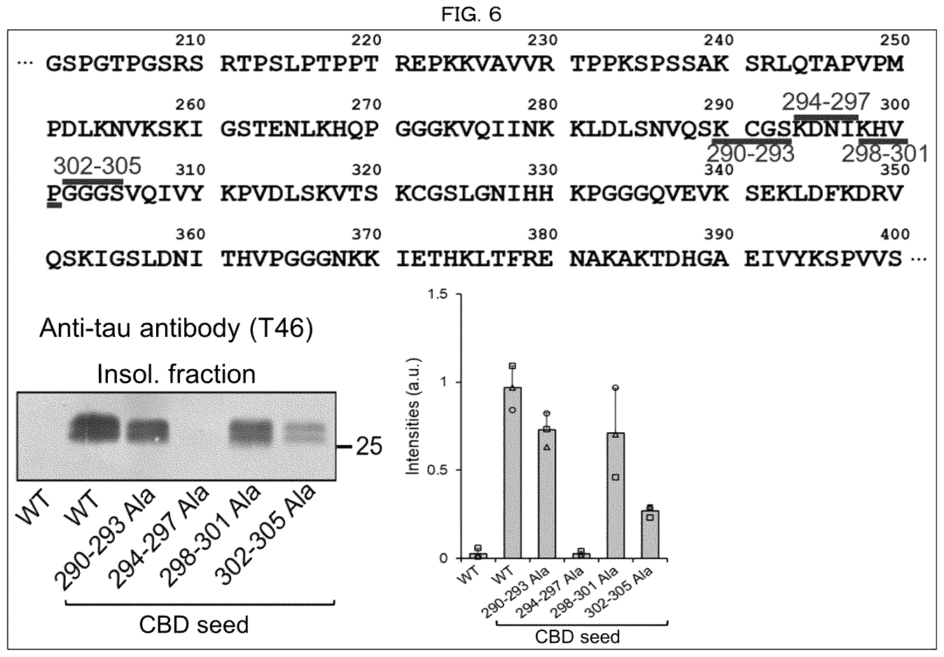

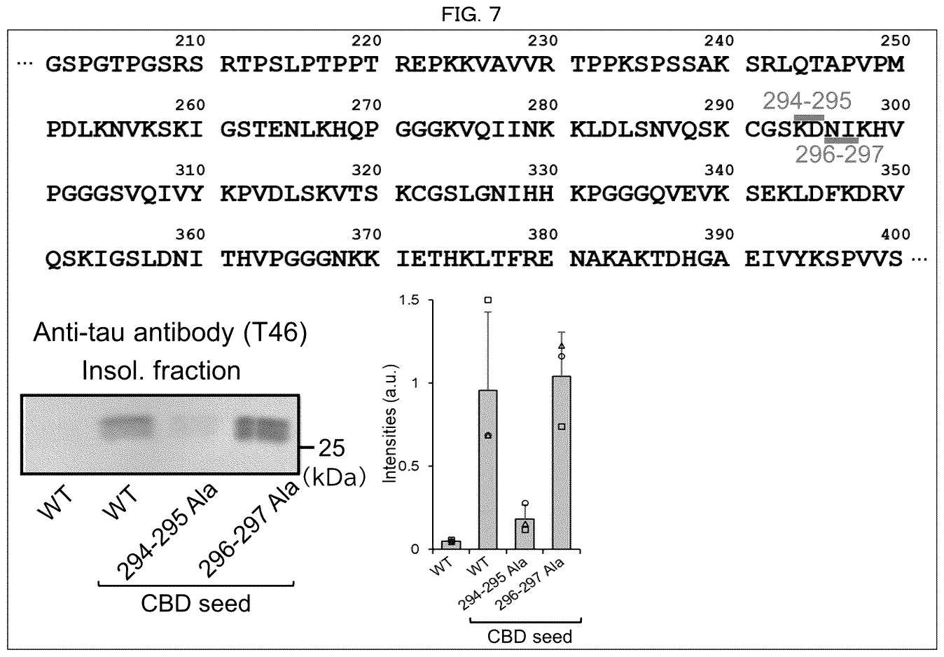

For CBD, 290-305 aa was focused on, and cellular expression vectors were constructed for tau in which substitution with Ala was carried out in groups of four amino acids, i.e., “290-293 Ala”, “294-297 Ala”, “298-301 Ala”, and “302-305 Ala”. These variants were expressed in SH-SY5Y cells, aggregation was induced by CBD seeds, and the cells were cultured, after which the bands in the insol. fractions were quantified. As a result, a particularly remarkable decrease in aggregation was observed in “294-297 Ala” (FIG. 6). Subsequently, expression vectors for “294-295 Ala” and “296-297 Ala”, into which said sequence was further subdivided, were constructed, and a similar experiment was conducted. As a result, a decrease in aggregation was observed in “294-295 Ala” (FIG. 7).

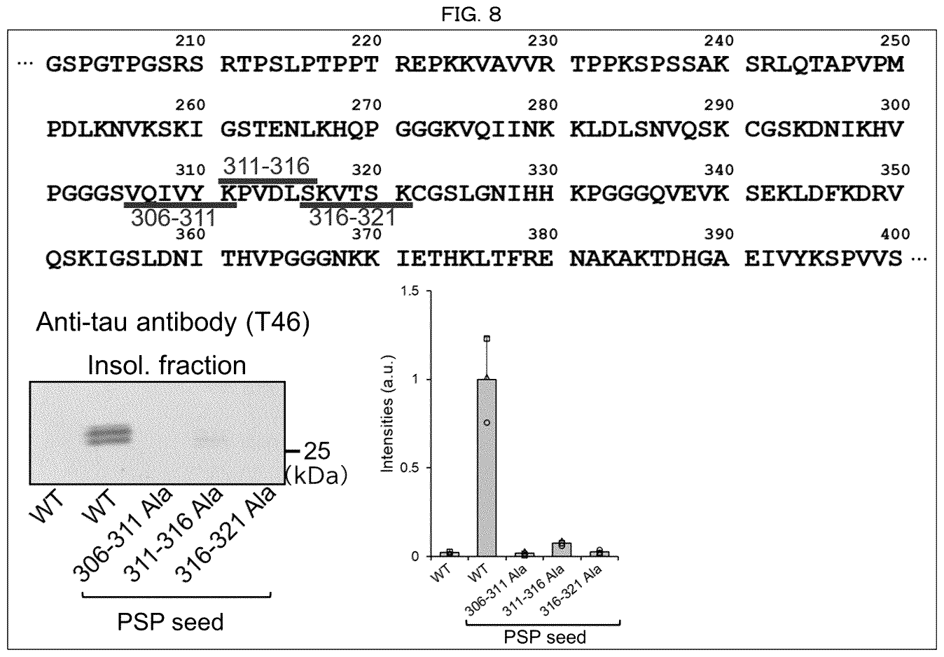

Next, for PSP, 306-321 aa was focused on, and vectors were constructed to express three variants, “306-311 Ala”, “311-316 Ala”, and “316-321 Ala” (FIG. 8). These variants were expressed in SH-SY5Y cells, aggregation was induced by PSP seeds, and the cells were cultured, after which the bands in the insol. fractions were quantified. As a result, a remarkable decrease in aggregation was observed in all of the variants. Among these, “306-311 Ala” showed decreased aggregation regardless of the type of seed.

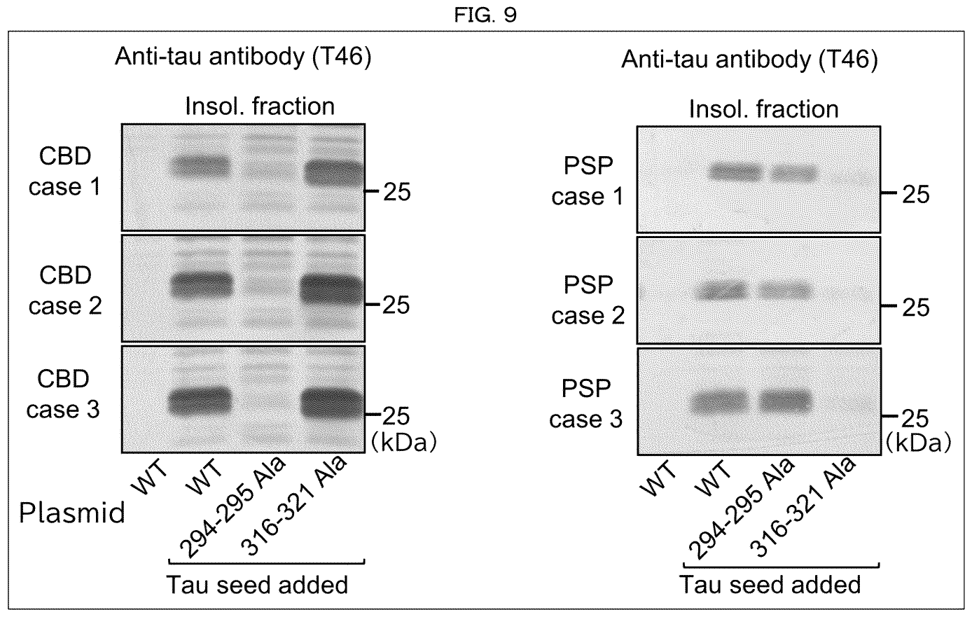

In summary, it is shown that “294-295 Ala” reduces the aggregation ability of tau in a CBD seed-specific manner, and “316-321 Ala” reduces the aggregation ability of tau in a PSP seed-specific manner (FIG. 9). SH-SY5Y cells expressing “294-295 Ala” and SH-SY5Y cells expressing “316-321 Ala” were cultured with the addition of CBD and PSP seeds, after which the insol. fractions were prepared, and the aggregates were quantified by Western blotting. As a result, in the system in which CBD seeds were added, decreased aggregation was observed only for “294-295 Ala”, and, in the system in which PSP seeds were added, decreased aggregation was observed only for “316-321 Ala”.

Therefore, “294-295 Ala” was determined to be a mutation that specifically decreases aggregation against CBD seeds, and “316-321 Ala” was determined to be a mutation that specifically decreases aggregation against PSP seeds.

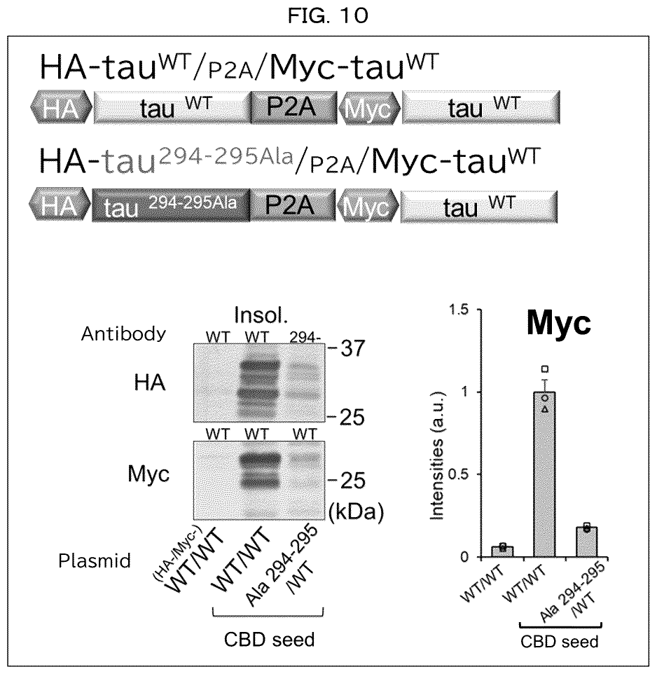

Next, to verify whether tau²⁹⁴⁻²⁹⁵ Ala inhibits aggregation of tau^{WT} induced by CBD seeds, “pcDNA3-HA-tau²⁹⁴⁻²⁹⁵ Ala/P2A/Myc-tau^{WT}” was constructed as a cellular co-expression vector (FIG. 10). CBD seeds were introduced into cells in which “pcDNA3-HA-tau^{WT}/P2A/Myc-tau^{WT}” was expressed and cells in which the newly constructed “pcDNA3-HA-tau²⁹⁴⁻²⁹⁵ Ala/P2A/Myc-tau^{WT}” was expressed, and the cells were cultured for 3 days. Thereafter, the insol. fractions were prepared in the same manner, and Western blotting was performed to quantify the aggregated Myc-tau^{WT} band. As a result, it was revealed that, in the sample in which tau^{294-295 Ala} was co-expressed, the aggregated Myc-tau^{WT} band was markedly reduced. This suggested that tau^{294-295Ala} interferes with and inhibits CBD seed aggregation of tau^{WT}, just as tau^{Δ368} did against AD seed aggregation.

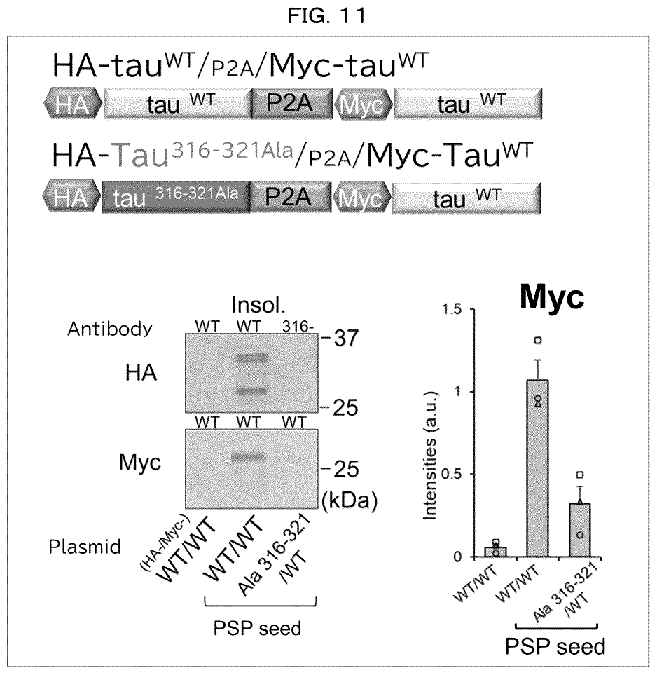

Similarly, to verify whether tau^{316-321 Ala} inhibits aggregation of tau^{WT} induced by PSP seeds, “pcDNA3-HA-tau^{316-321 Ala}/P2A/Myc-tau^{WT}” was constructed as a cellular co-expression vector (FIG. 11). PSP seeds were introduced into cells in which “pcDNA3-HA-tau^{WT}/P2A/Myc-tau^{WT}” was expressed and cells in which the newly constructed “pcDNA3-HA-tau^{316-321 Ala}/P2A/Myc-tau^{WT}” was expressed, and the cells were cultured for 3 days. Thereafter, the insol. fractions were prepared in the same manner, and Western blotting was performed to quantify the aggregated Myc-tau^{WT} band. As a result, it was revealed that, in the sample in which tau^{316-321 Ala} was co-expressed, a reduction in the aggregated Myc-tau^{WT} band was observed. This suggested that tau^{306-321 Ala} interferes with and inhibits PSP seed aggregation of tau^{WT}.

From the above, it was revealed that, when tau^{Δ368}, tau²⁹⁴⁻²⁹⁵ Ala, and tau^{316-321 Ala}, which are artificial variants of tau, are co-expressed intracellularly, aggregations of tau^{WT} induced by AD seeds, CBD seeds, and PSP seeds are suppressed, respectively. This indicates that tau into which an artificial mutation has been introduced interferes with and inhibits pathological tau aggregation, which is a novel finding that has never been reported before. A particularly important point of the present invention is that, by selecting the mutation to be introduced from among A368, 294-295 Ala, and 316-321 Ala, it is possible to achieve inhibition tailored to the form of tau aggregation in AD, CBD, and PSP.

For AD, which is a typical tauopathy, disease-modifying drugs targeting amyloid-β as well as the previous symptom-improving drugs have already been launched on the market; however, the development of disease-modifying drugs that act on tau is currently delayed. Based on the findings of the present Example, for example, expected is a treatment method of causing an adeno-associated virus (AAV) vector to carry information on the sequence of artificially mutated tau, and infecting a patient's brain in which tau aggregation is taking place with the adeno-associated virus (AAV) vector, thereby causing the artificially mutated tau expressed within the neuronal cells of the patient's brain to suppress tau aggregation. In this case, by examining the AAV serotype and the administration method, highly efficient and long-term administration of the artificially mutated tau, which is a “therapeutic protein”, into neuronal cells is possible in the form of sustained protein expression. In the future, it is also expected that, for example, therapeutic tau is created which can address, not a specific strain, but all tauopathies by introducing all of these three types of mutations.

1) Construction of Cellular Expression Plasmid Vector that Encodes Tau Variant

Using an expression plasmid pcDNA3 into which a cDNA sequence of a human tau fragment (243-441 aa) was inserted, a plasmid encoding a tau variant was prepared. Primers used in this procedure are shown below. Using these primers and the above-described wild-type vector as a template, various variant vectors were prepared with use of KOD Plus Mutagenesis Kit (Toyobo).

Primers for substituting specific regions of the tau sequence with Ala:

=tau 290-293 Ala=
For: GCCGCCGCTGCGAAGGATAATATCAAACACGTC
Rev: GGACTGGACGTTGCTAAGATC
=tau 294-297 Ala=
For: GCCGCCGCTGCGAAACACGTCCCGGGAGGCG
Rev: TGAGCCACACTTGGACTGGACG
=tau 298-301 Ala=
For: GCCGCCGCTGCGGGAGGCGGCAGTGTGCAAATAG
Rev: GATATTATCCTTTGAGCCACAC
=tau 302-305 Ala=
For: GCCGCCGCTGCGGTGCAAATAGTCTACAAACC
Rev: CGGGACGTGTTTGATATTATC
=tau 294-295 Ala=
For: GCCGCCAATATCAAACACGTCCCGGGAGGC
Rev: TGAGCCACACTTGGACTGGACG
=tau 296-297 Ala=
For: GCCGCCAAACACGTCCCGGGAGGCGGC
Rev: ATCCTTTGAGCCACACTTGGAC
=tau 306-311 Ala=
For: GCCGCCGCTGCGGCAGCT CCAGTTGACCTGAGCAAGGTGAC
Rev: ACTGCCGCCTCCCGGGACGTGTTTG
=tau 311-316 Ala=
For: GCCGCCGCTGCGGCAGCT AAGGTGACCTCCAAGTGTGGCTC
Rev: GTAGACTATTTGCACACTGCC
=tau 316-321 Ala=
For: GCCGCCGCTGCGGCAGCT TGTGGCTCATTAGGCAACATCC
Rev: CAGGTCAACTGGTTTGTAGAC

The primers for substituting specific regions of the tau sequence with Ala are as described above.

Next, to construct a co-expression vector using a P2A sequence, the P2A sequence of GSGATNFSLLKQAGDVEENPGP was inserted into the multiple cloning site region of pcDNA3. This was performed with use of KOD Plus Mutagenesis Kit, using the following primers.

=P2A insertion (step 1: insertion of the first
half, GSGATNFSLLK)=
For: GGAAGCGGAGCTACTAACTTCAGCCTGCTGAAGCAGTGGCGG
CCGCTCGAGTC
Rev: TGCTGGATATCTGCAGAATTC
=P2A insertion (step 2: insertion of the second
half, QAGDVEENPGP)=
For: CAGGCTGGAGACGTGGAGGAGAACCCTGGACCT
CAGTGGCGGCCGCTCGAGTC
Rev: CTTCAGCAGGCTGAAGTTAGTAGC

The primers for insertion of the P2A sequence are as described above.

Next, HA-tau and Myc-tagged tau were incorporated upstream and downstream of the P2A sequence, respectively. To incorporate HA-tau upstream of the P2A sequence, first, the pcDNA3-P2A vector was treated with restriction enzymes BamH1 (Toyobo, Cat. #BAH-111) and EcoR1 (Toyobo, Cat. #ECO-111) at 37° C. for 2 hours and linearized.

In addition, to prepare the HA-tau sequence as an insert (insertion fragment), PCR was performed using the following primers and PrimeSTAR GXL DNA polymerase (TaKaRa, Cat. #R050A).

=HA-tau fragment=
For: TACCGAGCTCGGATCCATGTACCCATACGATGTTCC
Rev: GATATCTGCAGAATTCCAAACCCTGCTTGGCCAG

The insert was cloned into the obtained vector using In-Fusion HD Cloning kit (Clontech, Cat. #639648).

To incorporate Myc-tau downstream of the P2A sequence, first, the pcDNA3-P2A vector was treated with a restriction enzyme Not1 (Toyobo, Cat. #NOT-111) at 37° C. for 2 hours and linearized.

In addition, to prepare the Myc-tau sequence as an insert (insertion fragment), PCR was performed using the following primers and PrimeSTAR GXL DNA polymerase.

=Myc-tau fragment=
For: ACCTCAGTGGCGGCCTATGGAGCAGAAACTCATCTC
Rev: TAGACTCGAGCGGCCTCACAAACCCTGCTTGGC

The insert was cloned into the obtained vector using In-Fusion HD Cloning kit (Clontech, Cat. #639648).

### 2) Preparation of Patient's Brain-Derived Tau Seeds

0.25 g of a brain from a patient biochemically and neuropathologically diagnosed with AD, CBD, or PSP was immersed in 1.0 mL of ice-cooled A68 buffer (10 mM Tris-HCl, pH 7.5/1 mM EGTA/10% sucrose/0.8 M NaCl) and homogenized on ice using a dounce-type homogenizer. Thereafter, ultrasonication was performed on ice for 1 minute using a TAITEC VP-050 ultrasonic processor (at an intensity of 30% PWM), and a homogenate was transferred to 1.5 mL tubes in equal amounts. Next, centrifugation was performed using a high-speed microcentrifuge under conditions of 3000×g for 15 minutes, the post-centrifugation supernatant was transferred to a new 1.5 mL tube, and this was used as a patient brain tau seed.

### 3) Cultured Cells

A human neuroblast SH-SY5Y strain (American Type Culture Collection, Cat. #CRL-2266) purchased from American Type Culture Collection was used. As a culture solution, DMEM (Dulbecco's modified eagle's medium nutrient mixture)/F-12HAM (Sigma-Aldrich, Cat. #D8062-500ML) supplemented with 10% (v/v) fetal bovine serum, a non-essential amino acid solution (MEM Non-Essential Amino Acids Solution (100X), ThermoFisher, Cat. #11140050), and a penicillin-streptomycin-glutamine solution (Penicillin-Streptomycin-Glutamine (100X), ThermoFisher, Cat. #10378016) was used, and culture was performed at 37° C. in a 5% CO₂ incubator (Thermo SCIENTIFIC). For the culture, a collagen-coated 6 cm dish (biocoat 6 cm dish, Corning, Cat. #356401) and a 24-well plate (biocoat 24 well plate, Corning, Cat. #356408) were used. Subculturing of the cells was performed by the following procedure in a state in which the cells reached 100% confluence. After the medium in the 6 cm dish was removed, the cells were washed with 2.0 mL of 1×PBS, and the 1×PBS was removed. Then, 0.5 mL of 0.25% trypsin was added, and the cells were kept warm at 37° C. for 5 minutes. Thereafter, 2.5 mL of fresh medium was further added to stop the reaction of trypsin, after which the cells were sufficiently suspended and seeded into a 6 cm dish containing 3 mL of medium.

4) Expression of Tau Plasmid and Introduction of Patient Brain Tau Seeds into Cells

In a 24-well plate, 8×10⁵ cells were seeded per well and cultured for 2 days. Thereafter, for plasmid introduction into the cells, a mixture solution of a WT expression plasmid (pcDNA3-tau) and X-treamGENE9 (Roche, Cat. #6365809001) was prepared. Specifically, Opti-MEM (ThermoFisher, Cat. #31985062), the plasmid, and X-treamGENE9 were gently mixed at a ratio of 20 μL: 0.2 μg: 0.6 μL and allowed to stand at room temperature for 15 minutes. Concurrently, for the introduction of patient brain tau seeds into the cells, a mixture solution of the patient brain seeds and Multifectam (Promega, Cat. #ETF5000) was prepared. Specifically, Opti-MEM, the patient brain tau seeds, and Multifectam were gently mixed at a ratio of 25 μL: 0.8 μL: 12.5 μL, allowed to stand at room temperature for 30 minutes, after which 12.5 L of Opti-MEM was added, and the mixture was allowed to stand for additional 5 minutes. Thereafter, the two types of mixture solutions were sequentially added dropwise into the culture solution in each well. The treated cells were incubated in a CO₂ incubator. After 24 hours, the culture solution was removed using an aspirator, 0.5 mL of fresh medium pre-warmed at 37° C. was added to each well, and the medium was exchanged. The cells were then returned to the CO₂ incubator and incubated for 48 hours.

### 5) Detection of Insoluble Tau by Western Blotting

The culture solution in each well was removed using an aspirator, and 10.5 mL of saline was added to detach and collect the cells from the plate. The cells were collected by centrifugation at 6,500 g for 10 minutes, and 150 μL of A68 buffer (10 mM Tris-HCl, pH 7.5/1 mM EGTA/10% sucrose/0.8 M NaCl) was added, followed by disruption of the cells by processing for 40 to 60 seconds using a TAITEC VP-050 ultrasonic processor (at an intensity of 25% PWM). After centrifugation at 100,000×g for 20 minutes (himac CS100GXL, Eppendorf-himac technologies), the supernatant and the pellets were separated. A68 buffer containing 1% Triton-X100 (t-octylphenoxypolyethoxyethanol, Sigma-Aldrich, Cat. #T6878) was added to the pellets, and the pellets were disrupted by ultrasonication for 15 seconds (at an intensity of 25% PWM). After centrifugation at 100,000 g for 20 minutes, the supernatant and the pellets were separated. A68 buffer containing 1% Sarkosyl (sodium N-lauroylsarcosinate, FUJIFILM Wako Pure Chemical Corporation, Cat. #194-10381) was added to the pellets, and the pellets were disrupted by processing for 15 seconds using the TAITEC VP-050 ultrasonic processor (at an intensity of 25% PWM). After centrifugation at 100,000 g for 20 minutes, the supernatant and the pellets were separated, and 30 μL of 2× SDS sample buffer containing 2% 2-mercaptoethanol was added to the pellets. Thereafter, the pellets were disrupted by ultrasonication for 10 seconds (at an intensity of 20% PWM), and the mixture was heat-treated at 100° C. for 5 minutes to obtain a surfactant-insoluble fraction (Insol. fraction).

The Insol. fraction was electrophoresed on a 15% polyacrylamide gel and then transferred to a PVDF membrane (Millipore) under conditions of 200 mA for 1 hour. The PVDF membrane was blocked in 1×PBS containing 3% gelatin (FUJIFILM Wako Pure Chemical Corporation, Cat. #077-03155) at room temperature for 10 minutes, and then reacted overnight at room temperature with primary antibodies (an antibody that recognizes tau: T46 antibody, 1:1,000 dilution, Thermo, Cat. #13-6400; an antibody that recognizes HA-tag: Anti-HA antibody, 1:1,000 dilution, Sigma-Aldrich, Cat. #H9658; an antibody that recognizes Myc-tag: Anti-Myc antibody, 1:1,000 dilution, Proteintech, Cat. #16286-1-AP) diluted with 1×PBS containing 10% bovine serum (CS: Bovine Serum, ThermoFisher, Cat. #16170-078) and 0.1% NaN3 (10% CS/1×PBS). Thereafter, the PVDF membrane was washed with 1×PBS, and reacted for 2 hours at room temperature with secondary antibodies (an antibody against the mouse-derived antibodies T46 and Anti-HA: Biotin-Goat anti mouse IgG, Vector, Cat. #BA-9200-1.5; an antibody against the rabbit-derived antibody Anti-Myc: Biotin-Goat anti rabbit IgG, Vector, Cat. #BA-1000-1.5) diluted with 10% CS/saline, followed by washing with 1×PBS. The PVDF membrane was reacted with a peroxidase-labeled avidin-biotin complex (ABC Standard Kit, Vector, Cat. #PK-4000) for 30 minutes, then washed with saline, and treated with 1×PBS containing 0.1% 3,3′-Diaminobenzidine (Sigma-Aldrich, Cat. #D8001-5G), 0.2 mg/mL nickel (II) chloride hexahydrate (FUJIFILM Wako Pure Chemical Corporation, Cat. #141-01045), and 0.05% H₂O₂ (hydrogen peroxide solution, Sigma-Aldrich, Cat. #13-1910-5) to develop protein bands on the membrane. The color development reaction was stopped by washing the PVDF membrane with tap water.

The present invention relates to a technique for suppressing tau aggregation and is very useful for, for example, tau aggregation-related research and medical applications.

1. A composition for suppression of tau aggregation, said composition comprising a variant of tau or a nucleic acid construct that expresses the variant.

2. The composition of claim 1, wherein the variant of the tau has at least one of mutations shown in the following (1) to (4):
(1) a mutation of an amino acid corresponding to a 368th amino acid (asparagine) in an amino acid sequence represented as an amino acid sequence (SEQ ID NO: 1) of wild-type tau;
(2) a mutation(s) of an amino acid(s) corresponding to a 294th amino acid (lysine) and/or a 295th amino acid (aspartic acid) in the amino acid sequence represented by SEQ ID NO: 1;
(3) a mutation(s) of an amino acid(s) corresponding to one amino acid, two amino acids, three amino acids, four amino acids, five amino acids, or all of six amino acids in a region (SKVTSK) consisting of 316th to 321st amino acids in the amino acid sequence represented by SEQ ID NO: 1; and
(4) a mutation(s) of an amino acid(s) corresponding to one amino acid, two amino acids, three amino acids, four amino acids, five amino acids, or all of six amino acids in a region (KPVDLS) consisting of 311th to 316th amino acids in the amino acid sequence represented by SEQ ID NO: 1.

3. The composition of claim 1, wherein said composition suppresses seed-dependent tau aggregation.

4. The composition of claim 3, wherein said composition suppresses tau aggregation dependent on a seed of Alzheimer's disease, suppresses tau aggregation dependent on a seed of corticobasal degeneration, or suppresses tau aggregation dependent on a seed of progressive supranuclear palsy.

5. The composition of claim 1, wherein said composition is for treating or preventing a tauopathy.

6. The composition of claim 2, wherein the variant of the tau is two or more types of variants of the tau selected from the group consisting of a variant having the mutation shown in (1) above, a variant having the mutation shown in (2) above, a variant having the mutation shown in (3) above, a variant shown in (4) above, and a variant shown in the following (5):
(5) a mutation(s) of an amino acid(s) corresponding to one amino acid, two amino acids, three amino acids, four amino acids, five amino acids, or all of six amino acids in a region (VQIVYK) consisting of 306th to 311th amino acids in the amino acid sequence represented by SEQ ID NO: 1.

7. A variant of tau having at least one of the following mutations (2) to (4):
(2) a mutation(s) of an amino acid(s) corresponding to a 294th amino acid (lysine) and/or a 295th amino acid (aspartic acid) in the amino acid sequence represented by SEQ ID NO: 1;
(3) a mutation(s) of an amino acid(s) corresponding to one amino acid, two amino acids, three amino acids, four amino acids, five amino acids, or all of six amino acids in a region (SKVTSK) consisting of 316th to 321st amino acids in the amino acid sequence represented by SEQ ID NO: 1; and
(4) a mutation(s) of an amino acid(s) corresponding to one amino acid, two amino acids, three amino acids, four amino acids, five amino acids, or all of six amino acids in a region (KPVDLS) consisting of 311th to 316th amino acids in the amino acid sequence represented by SEQ ID NO: 1.

8. A nucleic acid construct encoding the variant of tau of claim 7.

9. A method for suppressing seed-dependent tau aggregation, said method comprising a step of causing a variant of tau to co-exist with wild-type tau.

10. The method of claim 9, wherein:
the variant of the tau is derived from a composition for suppression of tau aggregation; and
the composition comprises a variant of tau or a nucleic acid construct that expresses the variant.

11. The method of claim 10, wherein the variant of the tau has at least one of mutations shown in the following (1) to (4):
(1) a mutation of an amino acid corresponding to a 368th amino acid (asparagine) in an amino acid sequence represented as an amino acid sequence (SEQ ID NO: 1) of wild-type tau;
(2) a mutation(s) of an amino acid(s) corresponding to a 294th amino acid (lysine) and/or a 295th amino acid (aspartic acid) in the amino acid sequence represented by SEQ ID NO: 1;
(3) a mutation(s) of an amino acid(s) corresponding to one amino acid, two amino acids, three amino acids, four amino acids, five amino acids, or all of six amino acids in a region (SKVTSK) consisting of 316th to 321st amino acids in the amino acid sequence represented by SEQ ID NO: 1; and
(4) a mutation(s) of an amino acid(s) corresponding to one amino acid, two amino acids, three amino acids, four amino acids, five amino acids, or all of six amino acids in a region (KPVDLS) consisting of 311th to 316th amino acids in the amino acid sequence represented by SEQ ID NO: 1.

12. The method of claim 9, wherein said composition suppresses tau aggregation dependent on a seed of Alzheimer's disease, suppresses tau aggregation dependent on a seed of corticobasal degeneration, or suppresses tau aggregation dependent on a seed of progressive supranuclear palsy.

13. The method of claim 9, wherein said method is carried out for a purpose of treating or preventing a tauopathy.

14. The method of claim 11, wherein the variant of the tau is two or more types of variants of the tau selected from the group consisting of a variant having the mutation shown in (1) above, a variant having the mutation shown in (2) above, a variant having the mutation shown in (3) above, a variant shown in (4) above, and a variant shown in the following (5):
(5) a mutation(s) of an amino acid(s) corresponding to one amino acid, two amino acids, three amino acids, four amino acids, five amino acids, or all of six amino acids in a region (VQIVYK) consisting of 306th to 311th amino acids in the amino acid sequence represented by SEQ ID NO: 1.

15. The method of claim 9, wherein the step of causing the variant of the tau to co-exist with wild-type tau is carried out in vitro, in a cell, or in vivo.
